SEQUENCE LISTING (2 sequences)

SEQ ID NO: 1 - DNA, 1201 bp, homo sapiens
  CDS (33)..(959)
agtcccagctcagagccgcaacctgcacagccatgcccgggcaagaactcaggacgctgaatggctctcagatgctcctggtgttgctggtgctctcgtggctgccgcatgggggcgccctgtctctggccgaggcgagccgcgcaagtttcccgggaccctcagagttgcacaccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgacctctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggaggcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 2 - protein, 308 aa, homo sapiens [sequence omitted]

(19) United States
(12) Patent Application Publication
(10) Pub. No.: US 2007/0212701 A1
(43) Pub. Date: Sep. 13, 2007

(54) Methods for diagnosing and treating ischemia and reperfusion injury and compositions thereof

(76) Inventors: Margot M. O'Toole, Newtonville, MA (US); Holly M. Legault, Concord, MA (US); Allan D. Kirk, Darnestown, MD (US)

Correspondence Address: FITZPATRICK CELLA HARPER & SCINTO, 30 ROCKEFELLER PLAZA, NEW YORK, NY, 10112, US

(21) Appl. No.: 11/519,294

(22) Filed: Sep. 11, 2006

Related U.S. Application Data
(62) Division of application No. 10/144,559, filed on May 13, 2002, now abandoned.
(60) Provisional application No. 60/290,529, filed on May 11, 2001.

(51) Int. Cl. C12Q 1/68; G01N 33/53

(52) U.S. Cl. 435/6; 435/7.71

(57) ABSTRACT

The present invention is directed to the identification of novel targets for therapeutic intervention and prevention of ischemia and reperfusion injury resulting from hypoxia, stroke, heart attack, chronic kidney failure or organ transplantation. In particular, the present invention is directed to the identification of novel targets for the prevention of reperfusion injury following organ transplantation. The present invention is further directed to methods of high-throughput screening for test compounds capable of inhibiting activity of proteins encoded by the novel targets by combining the test compounds and the protein and detecting binding. Moreover, the present invention is also directed to methods that can be used to assess the efficacy of test compounds and therapies for the ability to inhibit organ damage resulting from reperfusion injury. Methods for determining the prognosis of long term organ survival in a subject having an organ transplant are also described.

This application claims benefit of U.S. patent application Ser. No. 60/290,529 filed May 11, 2001.

### FIELD OF THE INVENTION

The present invention is directed to novel methods for diagnosis, treatment and prognosis of ischemia and reperfusion injury by identifying abnormally expressed genes. The present invention is further directed to therapeutic targets and to methods of screening and assessing test compounds for the intervention and prevention of organ damage resulting from reperfusion injury, particularly in relation to the field of organ transplantation.

### BACKGROUND OF THE INVENTION

In general terms, ischemia is described as insufficient circulation due to obstruction (e.g., arterial narrowing) of the blood supply while reperfusion injury is described as the damage caused by the subsequent reopening of the obstruction. The prototypical short-term response to ischemia and reperfusion consists of disruption of vascular homeostatic mechanisms, including vasoconstriction, thrombosis, and increased vascular permeability, as well as the activation of inflammatory responses which ultimately lead to fibrosis. The long-term pathophysiological responses to ischemia vary depending on the organ involved and the severity of the ischemia, e.g., hypoxia-ischemia in the brain has been linked to spreading depression, seizure, acute necrosis and delayed neurodegeneration. It is well known that long-lasting ischemia, wherein occlusion occurs for more than a few minutes, can bring about cell death and permanent organ damage.

Ischemia and reperfusion injury is a critical factor in determining the extent of organ damage following hypoxia, stroke, heart attack, chronic kidney failure and organ transplantation. Published studies have consequently focused on strategies to counteract the effects of the up-regulation of the early response genes associated with ischemic damage. Studies of hypoxia-ischemia in the mammalian brain have noted differing temporal and spatial patterns of up-regulation in the inducible transcription factors, Fos, Jun and Krox, in relation to morphological alterations and cell death in neural tissue. Herdegen, T. and Leah, J. D. (1998) Brain Res. Rev. 28L 370-490.

In the field of organ transplantation, donor organs subjected to prolonged ischemia suffer from reperfusion injury. Ischemia and reperfusion injury relating to transplantation is believed to be a major factor in initiating the cascade of responses that result in episodes of acute kidney rejection. These episodes, in turn, are widely believed to be the single most critical factor in the length of graft survival. A review of kidney transplants from cadavers in the United States found a strong correlation between the incidence of acute rejection and graft half life. Harisharan et al. (2000) New England J. of Med. 342(9):605. While progress has been made in lengthening the survival time of cadaver grafts which are free of acute rejection, no progress has been made in lengthening the survival time of cadaver grafts having episodes of acute rejection. This information, coupled with the fact that cadaver kidneys have a relatively poor survival time (62% versus 77% of living donors at 5 years, Transplant Patient DataSource. (2000, Feb. 16). Richmond, Va.: United Network for Organ Sharing) have led to the theory that ischemia damage is one of the critical factors in initiating acute rejection episodes and therefore graft rejection in general.

Current therapies developed to treat ischemia and reperfusion injury include administration by cyclosporine and trimetazidine, muromonab-CD3 (OKT3 monoclonal antibody), mycophenolate mofetil, tacrolimus or induction therapy, among other therapies well known in the art. As mentioned above however, these therapies have been unable to improve the survival rates of recipients of cadaver grafts suffering from episodes of acute rejection. Moreover, ongoing studies of organ transplants have focused on inflammatory responses triggered by ischemia and reperfusion injury with limited success in elucidating or modulating the earlier mechanisms behind ischemia and reperfusion injury. A study by Ritter et al. suggested that inhibition of the pro-inflammatory cytokines, e.g., TNF-α, would increase early expression of reporter genes to alleviate the inflammatory response, and thereby acute rejection. Ritter et al. (2000) Gene Therapy 7(14):1238-43. These studies have failed to distinguish the genes involved in ischemia and reperfusion apart from the triggered immunological response.

The nature and variability of ischemic injury as expressed in different animal models, different patients and different tissues, has proven to be a challenge in characterizing the phenomenon and in developing further methods for therapeutic intervention and prevention. Ischemia and reperfusion injury involve the complex interplay of numerous regulatory and inflammatory mechanisms. The present invention therefore addresses these issues by identifying abnormally or differentially expressed genes which may act as targets for modulation of ischemia and reperfusion injury. The present invention provides a number of novel therapeutic targets for blocking the effects of reperfusion injury, including in particular, orphan receptors that can be the focus of a search for inhibitors. This invention is further directed to a panel of markers for use in clinical testing of novel transplantation therapies. Unless otherwise noted in the application, the accession numbers provided refer to Genbank accession numbers, which can be found at http//www.ncbi.nlm.nih.gov.

### SUMMARY OF THE INVENTION

In one embodiment, the invention provides a method of assessing the efficacy of a test compound for inhibiting organ damage resulting from reperfusion in a subject by comparing the level of expression of a marker listed in Tables 3-7 in a first sample obtained from the subject, where the first sample is exposed to the test compound, to a second sample not exposed to the test compound, and where a substantially decreased level of expression of the marker in the first sample relative to the second sample, is an indication that the test compound is efficacious for inhibiting organ damage in the subject. In a preferred embodiment, the first and second samples are portions of a single sample obtained from the subject, and the level of expression in the first sample approximates the level of expression in a control sample. In a further preferred embodiment, the method is used to assess the efficacy of a test compound for inhibiting organ damage resulting from reperfusion. In a still further preferred embodiment, the reperfusion results from an organ transplantation, and in particular, a kidney transplantation. Alternatively, the samples may be collected from urine. In preferred embodiments, the marker is selected from the markers listed in either Tables 4, 6 or 7.

In another embodiment, the invention provides a method of assessing the efficacy of a therapy for inhibiting organ damage resulting from reperfusion in a subject by comparing expression of a marker listed in Tables 3-7 in a first sample obtained from the subject, prior to providing at least a portion of the therapy, to expression of the marker in a second sample following provision of the portion of the therapy, where a substantially decreased level of expression of the marker in the second sample relative to the first sample, is an indication that the test compound is efficacious for inhibiting organ damage in the subject. In a preferred embodiment, the level of expression of the marker in the second sample approximates the level of expression of the marker in a control sample.

The present invention also provides high-throughput screening for test compounds capable of inhibiting activity of a protein encoded by a marker listed in Tables 3-7 by combining the test compounds and the protein and then detecting binding of one of the test compounds to the protein, relative to other test compounds. In one embodiment, the protein encoded by a marker listed in Tables 3-7 that is used in the screening method is the TGF-beta family protein member, which is encoded by the nucleic acid sequence set forth in SEQ ID NO:1 (GenBank Accession No. AB000584). The encoded TGF-beta family protein amino acid sequence is set forth in SEQ ID NO:2. In a preferred embodiment, the selected test compound prevents binding of the protein with a bioactive agent selected from naturally occurring compounds, biomolecules, proteins, peptides, oligopeptides, polysaccharides, nucleotides or polynucleotides. More preferably, the bioactive agent is either a biomolecule or a polynucleotide. In an alternative embodiment, the step of detecting binding is conducted by utilizing surface plasmon resonance. In another preferred embodiment, the test compounds are bioactive agents selected from the group consisting of naturally occurring compounds, biomolecules, proteins, peptides, oligopeptides, polysaccharides, nucleotides or polynucleotides. Alternatively, the test compounds are small molecules. In another preferred embodiment, the method of high-throughput screening is conducted using markers selected from any of Tables 4, 6 or 7.

In yet another embodiment, the invention provides a method of high-throughput screening for test compounds capable of inhibiting organ damage resulting from reperfusion by combining a protein encoded by a marker listed in Tables 3-7, a specific factor which binds to the protein and the test compounds, and then selecting one test compound which prevents binding of the protein and the specific factor. In one embodiment, the protein encoded by a marker listed in Tables 3-7 that is used in the screening method is the TGF-beta family protein member, which is encoded by the nucleic acid sequence set forth in SEQ ID NO:1 (GenBank Accession No. AB000584). The encoded TGF-beta family protein amino acid sequence is set forth in SEQ ID NO:2. In a preferred embodiment, the step of selecting one test compound consists of detecting binding of the test compound to the protein. Alternatively, the step of selecting may consist of detecting binding of one of the test compounds to the specific factor. In a further preferred embodiment, the test compounds may be small molecules or bioactive agents. In a most preferred embodiment, the step of selecting utilizes surface plasmon resonance. In a still further preferred embodiment, the method uses markers listed in any of Tables 4, 6 or 7.

In another embodiment, the invention provides a method of screening for a test compound capable of interfering with the binding of a protein encoded by a marker listed in Tables 3-7 and a specific factor which binds to the protein by combining the protein, a test compound and the specific factor which binds to the protein, and determining the binding of the protein and the specific factor. In one embodiment, the protein encoded by a marker listed in Tables 3-7 that is used in the screening method is the TGF-beta family protein member, which is encoded by the nucleic acid sequence set forth in SEQ ID NO:1 (GenBank Accession No. AB000584). The encoded TGF-beta family protein amino acid sequence is set forth in SEQ ID NO:2. Preferably, the specific factor is a substrate for the protein. Alternatively, the specific factor is a ligand for the protein, and more preferably the ligand is a polynucleotide. In another preferred embodiment, the specific factor is a ligand and the protein is a cell surface receptor. In an alternative embodiment, the test compound is a small molecule or a bioactive agent, and is most preferably a protein. In a still further preferred embodiment, the method uses markers listed in any of Tables 4, 6 or 7.

In yet another embodiment, the invention provides a method of screening test compounds for inhibitors of organ damage resulting from reperfusion by obtaining a sample containing cells, by separately maintaining aliquots of the sample in the presence of a plurality of test compounds, and by comparing the expression levels of a marker selected from Tables 3-7 in each of the aliquots, and selecting a test compound which induces a substantially decreased level of expression of the marker in the aliquot containing that test compound, relative to other test compounds. In one embodiment, the protein encoded by a marker listed in Tables 3-7 that is used in the screening method is the TGF-beta family protein member, which is encoded by the nucleic acid sequence set forth in SEQ ID NO:1 (GenBank Accession No. AB000584). The encoded TGF-beta family protein amino acid sequence is set forth in SEQ ID NO:2. Preferably, the test compounds are small molecules selected from libraries consisting of spatially addressable parallel solid phase or solution phase libraries or synthetic libraries made from deconvolution, ‘one-bead one-compound’ methods or by affinity chromatography selection. Alternatively, the test compounds are bioactive agents such as naturally occurring compounds, biomolecules, proteins, peptides, oligopeptides, polysaccharides, nucleotides and polynucleotides. More preferably, the test compounds are proteins. In a still further preferred embodiment, the selected test compound induces an expression level in the marker that approximate a normal level of expression. In another preferred embodiment, the sample is collected from ischemic tissue, and the sample is taken after organ transplantation.

In another embodiment, the invention includes a kit for determining the prognosis for long term organ survival in a subject having an organ transplant, the kit having a nucleic acid probe where the probe specifically binds to a transcribed polynucleotide corresponding to a marker selected from the group consisting of markers listed in Tables 3-7.

In still another embodiment, the invention provides a kit for assessing the suitability of each of a plurality of compounds for inhibiting organ damage resulting from reperfusion in a subject, the kit having the plurality of compounds and a reagent for assessing expression of a marker selected from Tables 3-7.

In another embodiment, the invention includes a kit for determining the prognosis for long term organ survival in a subject having an organ transplant, the kit having an antibody which specifically binds with a protein corresponding to a marker selected from the group consisting of markers listed in Tables 3-7.

In yet another embodiment, the invention provides a method of modulating a level of expression of a marker selected from Tables 3-7 by providing to ischemic cells of a subject an antisense oligonucleotide complementary to a polynucleotide corresponding to the marker.

In another embodiment, the invention includes a method of modulating a level of expression of a marker selected from Tables 3-7 by providing to ischemic cells of a subject a protein. In a preferred embodiment, the protein is provided to the cells by providing a vector having a polynucleotide encoding the protein to the cells.

In yet another embodiment, the invention provides a method of modulating a level of expression of a marker selected from Tables 3-7 by providing to ischemic cells of the subject an antibody. Preferably, a therapeutic moiety is conjugated to the antibody.

In another embodiment, the invention includes a method of localizing a therapeutic moiety to ischemic tissue by exposing the tissue to an antibody which is specific to a protein encoded by a marker listed in Tables 3-7. In an alternative embodiment, the tissue is exposed to a plurality of antibodies which are each specific to a protein encoded by a marker listed in Tables 3-7.

In yet another embodiment, the invention provides a method of screening for a test compound capable of modulating the activity of a protein encoded by a marker listed in Tables 3-7 by combining the protein and the test compound, and determining the effect of the test compound on the therapeutic efficacy of the protein.

In still another embodiment, the invention provides a reagent having a protein encoded by a marker selected from Tables 3-7, where the reagent is utilized in high-throughput screening assays for inhibition of organ damage resulting from reperfusion. In another embodiment, the invention provides a biochip having a marker from Tables 3-7, where the biochip is utilized in high-throughput screening assays for inhibition of organ damage resulting from reperfusion.

In yet another embodiment, the invention provides a composition capable of modulating reperfusion injury in a subject by comprising a protein encoded by a marker listed in Tables 3-7 and a pharmaceutically acceptable carrier.

In another embodiment, the invention provides a biochip having a panel of markers selected from Tables 3-7, and preferably the markers are selected for subjects suspected of having severe reperfusion injury. In a more preferred embodiment, the biochip has markers are selected for subjects having undergone organ transplantation, particularly for kidney transplantation.

In yet another embodiment, the invention includes a method of determining the severity of reperfusion injury in a subject, by comparing the level of expression of a marker in a sample from the subject to a normal level of expression of the marker in a control sample, where the marker is listed in Tables 3-7 and where an abnormal increase in the level of expression of the marker in the sample from the subject relative to the normal level is an indication that the subject is suffering from severe reperfusion injury. Preferably, the marker corresponds to a transcribed polynucleotide or a portion thereof. In a more preferred embodiment, the sample is collected from tissue of an allograft organ such as a kidney allograft. Even more preferably, the sample is collected from kidney tissue after transplantation, and most preferably the control sample is collected from kidney tissue of the subject prior to transplantation and the abnormal increase is a factor of at least about 2. In an alternative embodiment, the sample is collected from urine. In a further preferred embodiment, the level of expression of the marker in the sample is assessed by detecting the presence in the sample of a protein corresponding to the marker. In a still further preferred embodiment, the presence of the protein is detected using a reagent which specifically binds with the protein, and more preferably the reagent includes an antibody or fragments thereof. Alternatively, the invention provides that the level of expression of the marker in the sample may be assessed by detecting the presence in the sample of a transcribed polynucleotide or portion thereof, where the transcribed polynucleotide comprises the marker. The transcribed polynucleotide may be an mRNA or a cDNA. Furthermore, the level of expression of the marker in the sample may be assessed by detecting the presence in the sample of a transcribed polynucleotide or a portion thereof which hybridizes with a labeled probe under stringent conditions, where the transcribed polynucleotide comprises the marker.

In another embodiment, the invention provides for a method of determining the severity of reperfusion injury in a subject by comparing the level of expression in a sample of the subject of each of a panel of markers independently selected from Tables 3-7, to the normal level of expression of the panel in a control sample, where the level of expression of the panel of markers is abnormally increased relative to the corresponding normal level of expression of the panel of markers, indicating that the subject is suffering from severe reperfusion injury. Preferably, the panel of markers has at least 5 markers. In a more preferred embodiment, the sample is collected from tissue of an allograft organ such as a kidney allograft. In a most preferred embodiment, the sample is collected from kidney tissue after transplantation, and in an even more preferred embodiment, and the control sample is from kidney tissue prior to transplantation. In a still further preferred embodiment, the panel of markers comprises markers from Table 3.

In another embodiment, the invention includes a method for determining the prognosis for long term organ survival in a subject having an organ transplant by comparing the level of expression of a marker in a sample to a normal level of expression in a control sample, where the marker is selected from Tables 3-7 and where an abnormal increase in the level of expression of the marker in the sample from the subject and the normal level is an indication that the subject has a poor prognosis for long term organ survival. In a preferred embodiment, at least 5 markers are selected from Tables 3-7. In a still further preferred embodiment, at least one marker corresponds to a transcribed polynucleotide or portion thereof. In a still further preferred embodiment, the sample is collected from kidney tissue after transplantation, and in an even more preferred embodiment, the control sample is collected from kidney tissue prior to transplantation. In another preferred embodiment, the abnormal increase is a factor of two.

In addition, the invention also provides novel therapeutic targets for the inhibition of organ damage resulting from reperfusion, where the therapeutic target comprises a marker listed in Tables 3-7. Alternatively, the novel therapeutic invention comprises a protein encoded by a marker listed in Tables 3-7.

Other features and advantages of the invention will be apparent from the following detailed description and claims.

### BRIEF DESCRIPTION OF THE DRAWINGS

FIG. 1 is a graphical representation of the expression levels of select genes listed in Table 5, as found on Day 3 after transplantation of kidneys in allografts and autografts in rhesus monkeys (see Example 1 below). Allograft genes are abnormally expressed relative to expression in autografts, which are expressed at normal range, suggesting that these genes may be involved in immunological response to antigen.

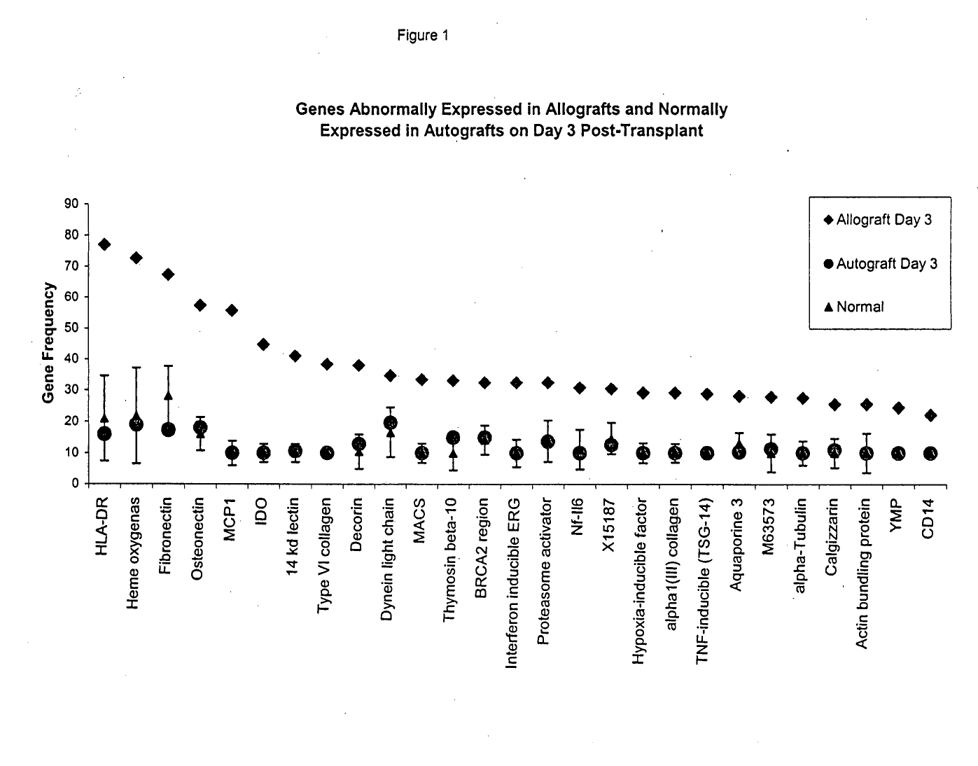

FIG. 2 is a graphical representation of the expression levels of the genes listed in Table 6, as found on Day 3 after transplantation of kidneys in allografts and autografts in rhesus monkeys (see Example 1 below). Allograft genes are expressed at abnormal levels with autografts showing a trend towards abnormal levels, suggesting that these genes may be independent of antigen and therefore active prior to immune response.

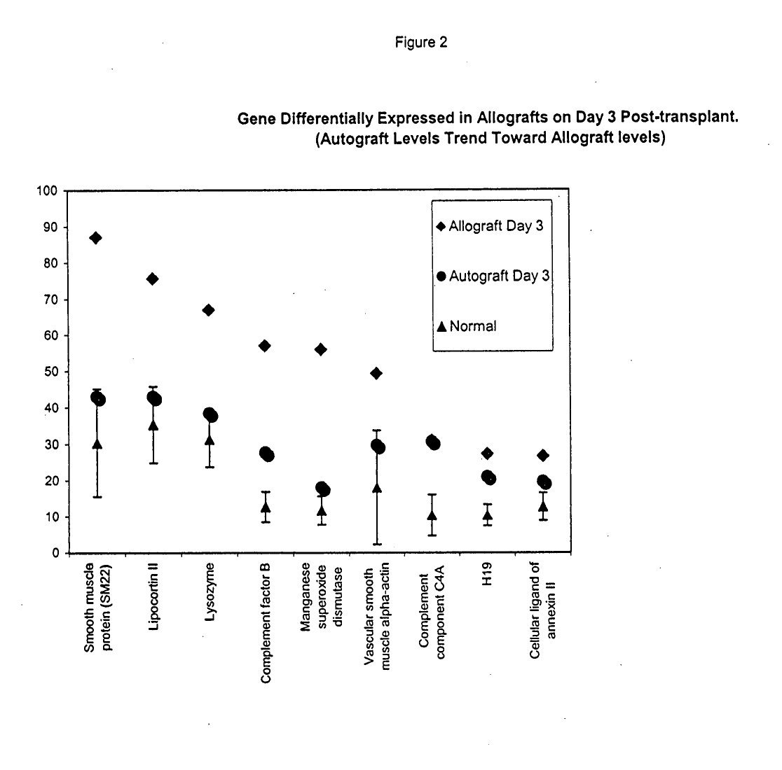

FIG. 3 is a graphical representation of the expression levels of the genes listed in Table 7, as found on Day 7 after transplantation of kidneys in allografts and autografts in rhesus monkeys (see Example 1 below). The figure indicates that a greater number of genes are now expressed at abnormal levels in both allografts and autografts.

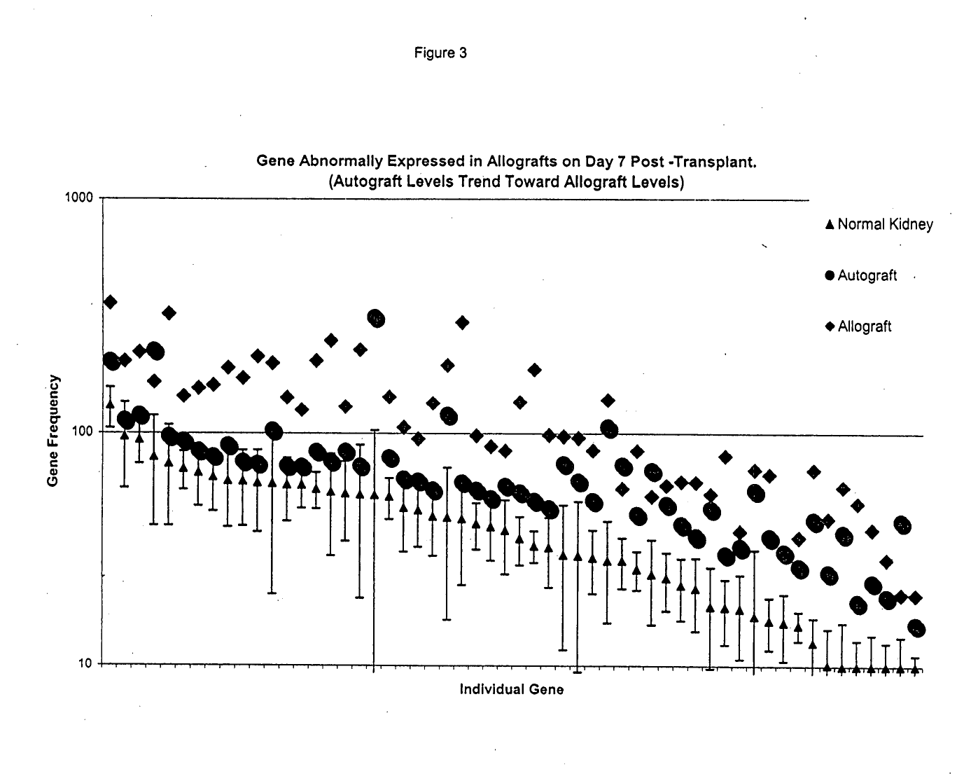

### DETAILED DESCRIPTION OF THE INVENTION

The present invention provides for the identification of novel targets for therapeutic intervention and prevention of ischemia and reperfusion injury resulting from hypoxia, stroke, heart attack, chronic kidney failure or organ transplantation. In particular, the present invention provides for the identification of novel therapeutic targets to be analyzed in high-throughput screening assays of test compounds capable of preventing ischemia and reperfusion injury following organ transplantation.

As shown in Examples 1 and 2 below, expression levels were recorded for both humans and rhesus monkey subjects prior to and following reperfusion during kidney transplantation. While rhesus monkey subjects were provided in the present invention for a more detailed analysis of ischemia and reperfusion injury resulting from kidney transplantation (Example 1), it is well-appreciated in the art that expression levels of genes in rhesus monkey may reflect expression levels from human subjects as well. Markers from other organisms may also be useful as animal models for the study of ischemia and reperfusion injury and for drug evaluation. Markers from other organisms may be obtained using the techniques outlined below.

In one aspect, the present invention is based on the identification of a number of genetic markers, set forth in Tables 3-7, which are expressed at abnormal or close-to-abnormal levels in ischemic samples of allografts and autografts, relative to normal untransplanted kidney samples. These markers may in turn be components of novel therapeutic targets for intervention in the development of reperfusion injury. The rhesus monkey is a well-accepted primate model for ischemia and reperfusion injury, and genes which are significant in reperfusion injury following organ transplantation in rhesus monkeys will likely play a role in human reperfusion injury. Consequently, kidney tissue from rhesus monkey was screened against a panel of 6,800 human genes. From a total of six rhesus monkeys, three received allograft kidney transplants and three received autograft kidney transplants (see Example 1). The expression levels of genes that were abnormally regulated between normal and allo- or auto-transplants at different stages post-reperfusion, are set forth in Tables 3 and 5-7, where the expression levels are provided as frequency of detected transcripts per million. In general, Tables 3 and 5-7 provide gene markers which were expressed at abnormal increased levels in allografts and close-to-abnormal increased levels in autografts. These genes may be a component in novel therapeutic targets for the treatment and prevention of ischemia and reperfusion injury.

In addition to primate data, biopsies from five human kidney transplants were collected from pre- and post-reperfusion and cadaveric samples to analyze differences in expression levels. As with the rhesus samples, the biopsies were screened against the panel of human markers to determine markers which were differentially expressed between normal samples and transplant samples. Comparison of the resulting genes was compared to genes in the rhesus monkey and resulted in five shared genes that were not previously linked to the pathophysiology of ischemia or reperfusion injury. The levels of expression for these five genes from either pre-reperfusion, post-reperfusion or cadaveric samples are provided in Table 4.

Included among the genes used to screen ischemic versus normal tissue in the human panel were several genes known in the art to be implicated in ischemia and reperfusion injury as listed in Tables 1 and 2. These genes served as an internal control in the primate model (Table 1) and human model (Table 2). Genes listed in Tables 1 or 2 were found to be increased in expression in the post-reperfusion kidney tissue as opposed to normal tissue, thus validating the method as a means for identifying significant genes involved in the pathophysiology of ischemia and reperfusion injury. Correspondingly, the genes which are known in the art to be linked to ischemia and reperfusion injury may also serve as validation in expression studies for ischemia and reperfusion injury. Moreover, the genes listed in Tables 3-7 that are expressed at abnormal or close-to-abnormal levels have not been previously associated with ischemia or reperfusion injury.

Accordingly, the present invention pertains to the use of the genes set forth in Tables 3-7, the corresponding mRNA transcripts, and the encoded polypeptides as markers for the presence of ischemia or reperfusion injury. Moreover, the use of expression profiles of these genes may indicate a risk of organ damage resulting from ischemia or reperfusion injury. With respect to organ transplantation, these markers are further useful to correlate differences in levels of expression with a poor or favorable prognosis for long-term organ survival. In particular, the present invention is directed to the genes set forth in Table 4, 6 and 7. Panels of the markers can be conveniently arrayed on solid supports, i.e., biochips for use in kits. Markers can also be useful for assessing the efficacy of a treatment or therapy of ischemia or reperfusion injury.

In one aspect, the invention provides markers whose level of expression, which signifies their quantity or activity, is correlated with the presence of ischemia or reperfusion injury. The markers of the invention may be nucleic acid molecules (e.g., DNA, cDNA or mRNA) or peptide(s). Preferably the invention is performed by detecting the presence of a transcribed polynucleotide or a portion thereof, wherein the transcribed polynucleotide comprises the marker. Alternatively, detection may be performed by detecting the presence of a protein which corresponds to the marker. The markers of the invention are typically increased to abnormal or close-to-abnormal levels of quantity or activity in ischemic tissue as compared to normal tissue.

In another aspect of the invention, the expression levels of genes are determined in a particular subject sample for which either diagnosis or prognosis information is desired. The level of expression of a number of genes simultaneously provides an expression profile, which is essentially a “fingerprint” of the activity of a gene or plurality of genes that is unique to the state of the cell. Comparison of relative levels of expression have been found to be indicative of the severity of reperfusion injury, which in the field of organ transplantation is correlated to the onset of acute rejection, and as such permits for diagnostic and prognostic analysis. Moreover, by comparing relative expression profiles of reperfusion injury from tissue samples taken at different points in time, e.g., pre- and post-reperfusion and several days after reperfusion, information regarding which genes are important in each of these stages is obtained. The identification of gene markers that are abnormally expressed in ischemic tissue versus normal tissue, as well as abnormal expression of genes during severe reperfusion injury ischemia, allows the use of this invention in a number of ways. For example, in the field of organ transplantation, comparison of expression profiles may provide a method for providing a prognosis for long term organ survival. In another example mentioned above, the evaluation of a particular treatment regime may be evaluated as to whether a particular drug act to improve the long-term prognosis in a particular patient. The discovery of these differential expression patterns for individual genes allows for screening of test compounds with an eye to modulating a particular expression pattern; for example, screening can be done for compounds that will convert an expression profile for a poor prognosis to a better prognosis. This may be done by making biochips comprising sets of the significant ischemic genes, which can then be used in these screens. These methods can also be done on the protein basis; that is protein expression levels of the ischemia-associated proteins can be evaluated for diagnostic and prognostic purposes or to screen test compounds. In addition, the markers can be administered for gene therapy purposes, including the administration of antisense nucleic acids, or proteins (including antibodies and other modulators thereof) administered as therapeutic drugs.

For example, the gene designated ‘UNK_M32053’ is abnormally increased in expression level in allograft and autograft kidney tissues on day 3 after reperfusion, relative to control tissue. The presence of increased mRNA for this gene (and for other genes set forth in Tables 3-7), and also increased levels of the protein products of this gene (and other genes set forth in Tables 3-7) serve as markers for ischemia or reperfusion injury. Preferably, for the purposes of the present invention, increased levels of the markers of the invention are increases of an abnormal magnitude, wherein the level of expression is outside the standard deviation for normal levels of expression. Most preferably, the marker is increased relative to control samples by at least 2-, 3-, or 4-fold or more, although such factorial increases are unlikely at early stages of reperfusion injury. One skilled in the art will be cognizant of the fact that a preferred detection methodology is one in which the resulting detection values are above the minimum detection limit of the methodology.

Detection and measurement of the relative amount of a nucleic acid or peptide marker of the invention may be by any method known in the art (see, e.g., Sambrook, J., Fritsch, E. F., and Maniatis, T. Molecular Cloning: A Laboratory Manual. 2^{nd} ed., Cold Spring Harbor Laboratory, Cold Spring Harbor Laboratory Press, Cold Spring Harbor, N.Y. (1989), and Current Protocols in Molecular Biology, eds. Ausubel et al, John Wiley & Sons (1992)). Typical methodologies for detection of a transcribed polynucleotide include RNA extraction from a cell or tissue sample, followed by hybridization of a labeled probe (i.e., a complementary nucleic acid molecule) specific for the target RNA to the extracted RNA and detection of the probe (i.e., Northern blotting). Typical methodologies for peptide detection include protein extraction from a cell or tissue sample, followed by binding of a labeled probe (i.e., an antibody) specific for the target protein to the protein sample, and detection of the probe. The label group can be a radioisotope, a fluorescent compound, an enzyme, or an enzyme co-factor. Detection of specific peptide(s) and nucleic acid molecules may also be assessed by gel electrophoresis, column chromatography, direct sequencing, or quantitative PCR (in the case of nucleic acid molecules) among many other techniques well known to those skilled in the art.

In certain embodiments, the genes themselves (i.e., the DNA or cDNA) may serve as markers for ischemia and reperfusion injury. For example, an increase of nucleic acid corresponding to a gene (i.e., a gene from Tables 3-7), such as by duplication of the gene, may also be correlated with ischemia or reperfusion injury.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (i.e., a complementary DNA molecules), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

The invention also encompasses nucleic acid and peptide molecules which are structurally different from the molecules described above (i.e., which have a slight altered nucleic acid or amino acid sequence), but which have the same properties as the molecules above (e.g., encoded amino acid sequences, or which are changed only in nonessential amino acid residues). Such molecules include allelic variants, and are described in greater detail in subsection I.

In another aspect, the invention provides markers whose quantity or activity is correlated with different manifestations or severity of reperfusion injury, including, in the field of organ transplantation, the onset of acute rejection. These markers are increased in quantity or activity in ischemic tissue in a fashion that is positively correlated with the degree of severity of reperfusion injury, which may in turn indicate permanent organ damage. The subsequent level of expression may further be compared to different expression profiles of various stages or times post-reperfusion to confirm whether the subject has a matching profile. In yet another aspect, the invention provides markers whose quantity or activity is correlated with a risk in a subject for developing organ damage (or acute rejection) resulting from reperfusion. As mentioned above, in the field of organ transplantion, acute rejection is correlated with higher incidence of chronic rejection. These markers are increased in activity or quantity in direct correlation to the likelihood of the development of permanent organ damage in a subject.

Each marker may be considered individually, although it is within the scope of the invention to provide combinations of two or more markers for use in the methods and compositions of the invention to increase the confidence of the analysis. In another aspect, the invention provides panels of the markers of the invention. In a preferred embodiment, these panels of markers are selected such that the markers within any one panel share certain features. For example, the markers of a first panel may each exhibit at least a two-fold increase in quantity or activity in ischemic tissue, as shown in Table 6, as compared to samples from normal samples from the same subject prior to transplantation. Alternatively, markers of a second panel may each exhibit abnormal regulation in autografts, as shown in Table 7. A third panel may also be devised in which each marker is expressed at abnormal or close-to-abnormal levels in both humans and primates, as in Table 4. Similarly, different panels of markers may be composed of markers from different samples (i.e., kidney, spleen, node, brain, heart or urine), or may be selected to represent different stages of reperfusion injury after the causative event (i.e., 30-60 minutes after, three days after or seven days after reperfusion). Panels of the markers of the invention may be made by independently selecting markers from any of Tables 3-7, and may further be provided on biochips, as discussed below.

It will be appreciated by one skilled in the art that the panels of markers of the invention may conveniently be provided on solid supports, as a biochip. For example polynucleotides may be coupled to an array (e.g., a biochip using GENECHIP® for hybridization analysis), to a resin (e.g., a resin which can be packed into a column for column chromatography), or a matrix (e.g., a nitrocellulose matrix for Northern blot analysis). The immobilization of molecules complementary to the marker(s), either covalently or noncovalently, permits a discrete analysis of the presence or activity of each marker in a sample. In an array, for example, polynucleotides complementary to each member of a panel of markers may individually be attached to different, known locations on the array. The array may be hybridized with, for example, polynucleotides extracted from a kidney sample from a subject. The hybridization of polynucleotides from the sample with the array at any location on the array can be detected, and thus the presence or quantity of the marker in the sample can be ascertained. In a preferred embodiment, an array based on a biochip is employed. Similarly, Western analyses may be performed on immobilized antibodies specific for different polypeptide markers hybridized to a protein sample from a subject.

It will also be apparent to one skilled in the art that the entire marker protein or nucleic acid molecule need not be conjugated to the biochip support; a portion of the marker of sufficient length for detection purposes (i.e., for hybridization) will suffice. For example, a portion of the marker that is 7, 10, 15, 20, 25, 30, 35, 40, 45, 50, 55, 60, 65, 70, 75, 100 or more nucleotides or amino acids in length may be sufficient for detection purposes.

The nucleic acid and peptide markers of the invention may be isolated from any tissue or cell of a subject. In a preferred embodiment, the tissue is from brain, heart, spleen, node or kidney. In a most preferred embodiment, the tissue is from kidney tissue. However, it will be apparent to one skilled in the art that other tissue samples, including bodily fluids such as blood or urine, may also serve as sources from which the markers of the invention may be assessed. The tissue samples containing one or more of the markers themselves may be useful in the methods of the invention, and one skilled in the art will be cognizant of the methods by which such samples may be conveniently obtained, stored and/or preserved.

Several markers were known prior to the invention to be associated with ischemia and reperfusion injury and are provided in Tables 1 and 2. These markers are not to be considered as markers of the invention. However, these markers may be conveniently used in combination with the markers of the invention (Tables 3-7) in the methods, panels and kits of the invention.

In another aspect, the invention provides methods of making an isolated hybridoma which produces an antibody useful for diagnosing a patient or animal with severe reperfusion injury. In this method, a protein corresponding to a marker of the invention is isolated (e.g., by purification from a cell in which it is expressed or by transcription and translation of a nucleic acid encoding the protein in vivo or in vitro using known methods). A vertebrate, preferably a mammal such as a mouse, rabbit or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment. The invention also includes hybridomas made by this method and antibodies made using such hybridomas.

The invention provides methods for determining the severity of reperfusion injury by isolating a sample from a subject (e.g., a sample containing kidney tissue or urine), detecting the presence, quantity and/or activity of one or more markers of the invention in the sample relative to a second sample from a normal sample. The levels of markers in the two samples are compared, and an abnormal increase in one or more markers in the test sample indicates severe reperfusion injury.

The invention also provides methods of assessing the efficacy of a test compound or therapy for inhibiting organ damage resulting from reperfusion in a subject. These methods involve isolating samples from a subject suffering from reperfusion injury, who is undergoing treatment or therapy, and detecting the presence, quantity, and/or activity of one or more markers of the invention in the first sample relative to a second sample. Where a test compound is administered, the first and second samples are preferably sub-portions of a single sample taken from the subject, wherein the first portion is exposed to the test compound and the second portion is not. In one aspect of this embodiment, the marker is expressed at a substantially decreased level in the first sample, relative to the second. Most preferably, the level of expression in the first sample approximates (i.e., is less than the standard deviation for normal samples) the level of expression in a third control sample, taken from a control sample of normal tissue.

Where the efficacy of a therapy is being assessed, the first sample obtained from the subject is preferably obtained prior to provision of at least a portion of the therapy, whereas the second sample is obtained following provision of the portion of the therapy. The levels of markers in the samples are compared, preferably against a third control sample as well, and correlated with the presence, risk of presence, or severity of reperfusion injury. Most preferably, the level of markers in the second sample approximates the level of expression of a third control sample. In the present invention, a substantially decreased level of expression of a marker indicates that the therapy is efficacious for inhibiting organ damage.

In addition, the invention provides methods of conducting high-throughput screening for test compounds capable of inhibiting activity of a proteins encoded by the novel markers of the invention. The method of high-throughput screening involves combining test compounds and the protein and determining whether the effect of the test compound on the encoded protein. Functional assays such as cytosensor microphysiometer, calcium flux assays such as FLIPR® (Molecular Devices Corp, Sunnyvale, Calif.), or the TUNEL assay may be employed to measure cellular activity, as discussed below. A variety of high-throughput functional assays well-known in the art may be used in combination to screen and/or study the reactivity of different types of activating test compounds, but since the coupling system is often difficult to predict a number of assays may need to be configured to detect a wide range of coupling mechanisms. A variety of fluorescence-based techniques are well-known in the art and are capable of high-throughput and ultra high throughput screening for activity, including but not limited, to BRET® or FRET® (both by Packard Instrument Co., Meriden, Conn.). A preferred high-throughput screening assay is provided by BIACORE® systems, which utilizes label-free surface plasmon resonance technology to detect binding between a variety of bioactive agents, as described in further detail below. The ability to screen a large volume and a variety of test compounds with great sensitivity permits for analysis of the therapeutic targets of the invention to further provide potential inhibitors of organ damage resulting from reperfusion. For example, where the marker encodes an orphan receptor with an unidentified ligand, high-throughput assays may be utilized to identify the ligand, and to further identify test compounds which prevent binding of the receptor to the ligand. The BIACORE® system may also be manipulated to detect binding of test compounds with individual components of the therapeutic target, to detect binding to either the encoded protein or to the ligand.

The invention also provides a method of screening test compounds for inhibitors of organ damage resulting from reperfusion, and to the pharmaceutical compositions comprising the test compounds. The method of screening comprises obtaining samples from subjects having undergone reperfusion, maintaining separate aliquots of the samples with a plurality of test compounds, and comparing expression of a marker in each of the aliquots to determine whether any of the test compounds provides a substantially decreased level of expression relative to samples with other test compounds or to an untreated sample. In addition, methods of screening may be devised by combining a test compound with a protein and thereby determining the effect of the test compound on the protein.

In addition, the invention is further directed to a method of screening for test compounds capable of interfering with the binding of a protein encoded by the markers of Tables 3-7 and a specific factor, by combining the test compound, protein, and specific factor together and determining whether binding of the specific factor and protein occurs. The test compound may be either small molecules or a bioactive agent. As discussed below, test compounds may be provided from a variety of libraries well known in the art.

Moreover, the invention is directed to pharmaceutical compositions comprising the test compound, or bioactive agent, which may further include a marker protein and/or nucleic acid of the invention (e.g., for those markers in Tables 3-7 that are increased in quantity or activity in ischemic tissue versus normal tissue), and can be formulated as described herein. Alternatively, these compositions may include an antibody that specifically binds to a marker protein of the invention and/or an antisense nucleic acid molecule that is complementary to a marker nucleic acid of the invention (e.g., for those markers that are increased in quantity in ischemic tissue) and can be formulated as described herein.

The invention further provides methods of modulating a level of expression of a marker of the invention, comprising administration to the ischemic cells of the subject a variety of compositions which correspond to the markers of Tables 3-7, including proteins or antisense oligonucleotides. The protein may be provided to the ischemic cells by further providing a vector comprising a polynucleotide encoding the protein to the cells. Alternatively, the expression levels of the markers of the invention may be modulated by providing an antibody, a plurality of antibodies or an antibody conjugated to a therapeutic moiety. Treatment with the antibody may further be localized to the ischemic tissue. In another aspect, the invention provides methods for localizing a therapeutic moiety to ischemic tissue comprising exposing the tissue to an antibody which is specific to a protein encoded by the markers of the invention. This method may therefore provide a means to inhibit or enhance expression of a specific gene corresponding to a marker listed in Tables 3-7. Where the gene is upregulated as a result of reperfusion injury, it is likely that inhibition or prevention of reperfusion would involve inhibiting expression of the upregulated gene.

In another aspect, the invention includes antibodies that are specific to proteins corresponding to markers of the invention. Preferably the antibodies are monoclonal, and most preferably, the antibodies are humanized, as per the description of antibodies below.

In still another aspect of the invention, the invention includes peptides or proteins which are encoded by the markers of the invention, and to compositions thereof.

The invention also provides kits for determining the prognosis for long term organ survival in a subject having an organ transplant, the kit comprising reagents for assessing expression of the markers of the invention. Preferably, the reagents may be an antibody or fragment thereof, wherein the antibody or fragment thereof specifically binds to a protein corresponding to a marker from Tables 3-7. Optionally, the kits may comprise a nucleic acid probe wherein the probe specifically binds to a transcribed polynucleotide corresponding to a marker selected from the group consisting of the markers listed in Tables 3-7.

The invention further provides kits for assessing the suitability of each of a plurality of compounds for inhibiting organ damage resulting from reperfusion in a subject. Such kits include a plurality of compounds to be tested, and a reagent (i.e. antibody specific to corresponding proteins of the invention) for assessing expression of a marker listed in Tables 3-7.

Modifications to the above-described compositions and methods of the invention, according to standard techniques, will be readily apparent to one skilled in the art and are meant to be encompassed by the invention.

To facilitate an understanding of the present invention, a number of terms and phrases are defined below:

As used herein, the term “modulation” includes, in its various grammatical forms (e.g., “modulated”, “modulation”, “modulating”, etc.), up-regulation, induction, stimulation, potentiation, and/or relief of inhibition, as well as inhibition and/or down-regulation.

As used herein, the terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are nonlimiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by nonnucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine©); guanine (G); thymine (T); and uracil (U) for guanine when the polynucleotide is RNA. This, the term “polynucleotide sequence” is the alphabetical representation of a polynucleotide molecule. This alphabetical representation can be inputted into databases in a computer having a central processing unit and used for bioinformatics applications such as functional genomics and homology searching.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of sill in the art, some of which are described herein.

A “gene product” includes an amino acid sequence (e.g., peptide or polypeptide) generated when a gene is transcribed and translated.

As used herein, a “polynucleotide corresponds to” another (a first) polynucleotide if it is related to the first polynucleotide by any of the following relationships:
1) the second polynucleotide comprises the first polynucleotide and the second polynucleotide encodes a gene product;
2) the second polynucleotide is 5′ or 3′ to the first polynucleotide in cDNA, RNA, genomic DNA, or fragments of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide. The first and second polynucleotide may be fragments of a gene encoding a gene product. The second polynucleotide may be an exon of the gene, while the first polynucleotide may be an intron of the gene;
3) the second polynucleotide is the complement of the first polynucleotide.

As used herein, the term, “transcribed” or “transcription” refers to the process by which genetic code information is transferred from one kind of nucleic acid to another, and refers in particular to the process by which a base sequence of mRNA is synthesized on a template of cDNA.

A “probe” when used in the context of polynucleotide manipulation includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set or primers” consisting of “upstream” and a “downstream” primer, and a catalyst of polymerization, such as a DNA polymerase, and typically a thermally stable polymerase enzyme. Methods for PCR are well known in the art, and are taught, for example, in MacPherson et al., IRL Press at Oxford University Press (1991)). All processes of producing replicate copies of a polynucleotide, such as PCR or gene cloning, are collectively referred to herein as “replication”. A primer can also be used as a probe in hybridization reactions, such as Southern or Northern blot analyses (see, e.g., Sambrook, J., Fritsch, E. F., and Maniatis, T. Molecular Cloning: A Laboratory Manual. 2^{nd}, ed., Cold Spring Harbor Laboratory, Cold Spring Harbor Laboratory Press, Cold Spring Harbor, N.Y., 1989).

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria (e.g., lambda phage). The library can then be probed for the specific cDNA (and thus mRNA) of interest.

A “gene delivery vehicle” includes a molecule that is capable of inserting one or more polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses and viral vectors, such as baculovirus, adenovirus, and retrovirus, bacteriophage, cosmid, plasmid, fungal vector and other recombination vehicles typically used in the art which have been described for replication and/or expression in a variety of eukaryotic and prokaryotic hosts. The gene delivery vehicles may be used for replication of the inserted polynucleotide, gene therapy, and polypeptide and protein expression.

A “vector” includes a self-replicating nucleic acid molecule that transfers an inserted polynucleotide into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above functions.

A “host cell” is intended to include any individual cell or cell culture which can be or has been a recipient for vectors or for the incorporation of exogenous nucleic acid molecules, polynucleotides and/or proteins. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, e.g., murine, rat, simian or human cells.

The term “genetically modified” includes a cell containing and/or expressing a foreign gene or nucleic acid sequence which in turn modifies the genotype or phenotype of the cell or its progeny. This term includes any addition, deletion, or disruption to a cell's endogenous nucleotides.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsch, E. F., and Maniatis, T. Molecular Cloning: A Laboratory Manual. 2^{nd}, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Differentially” or “abnormally” expressed, as applied to a gene, includes the differential production of mRNA transcribed from a gene or a protein product encoded by the gene. A differentially or abnormally expressed gene may be overexpressed or underexpressed as compared to the expression level of a normal or control cell. In one aspect, abnormal or differential expression refers to a level of expression that differs from normal levels of expression by one normal standard of deviation. In a preferred aspect, the differential is 2 times or higher than the expression level detected in a control sample, although the nature of ischemia and reperfusion injury at earlier stages renders such a magnitude less likely (Table 6 provides a listing of genes differentially expressed by at least two fold in allografts). The term “differentially” or “abnormally” expressed also includes nucleotide sequences in a cell or tissue that are expressed where silent in a control cell or not expressed where expressed in a control cell.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, there or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. The present invention also includes polynucleotides capable of hybridizing under reduced stringency conditions, more preferably stringent conditions, and most preferably highly stringent conditions, to polynucleotides described herein. Examples of stringency conditions are shown in Table A below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE A
Stringency Conditions

| Stringency | Polynucleotide | Hybrid | Hybridization Temperature | Wash Temperature |
|---|---|---|---|---|
| Condition | Hybrid | Length (bp)^{I} | and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + | | | | |
| # C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1xSSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, 1995, F. M. Ausubel et al., eds., John Wiley & Sons, Inc., sections 2.10 and 6.3-6.4, incorporated herein by reference.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

An “antibody” includes an immunoglobulin molecule capable of binding an epitope present on an antigen. As used herein, the term encompasses not only intact immunoglobulin molecules such as monoclonal and polyclonal antibodies, but also anti-idiotypic antibodies, mutants, fragments, fusion proteins, bi-specific antibodies, humanized proteins, and modifications of the immunoglobulin molecule that comprises an antigen recognition site of the required specificity.

As used herein, the term “ischemic” refers to cells, tissues or samples from a subject after the occurrence of ischemia or reperfusion, wherein the cell, tissue or sample has been affected by reperfusion resulting from any of a number of causative events: hypoxia, stroke, heart attack, kidney failure, organ transplantation and other well-known causative events. As used herein, the term “normal” refers to cells, tissues or other such samples taken either pre-reperfusion or from a subject who has not suffered the causative event resulting in reperfusion. Control samples of the present invention are taken from normal samples. As used herein, a “normal level of expression” refers to the level of expression associated with normal samples thereof. Preferred tissue (and cell) samples are from kidney, spleen, node, brain, heart, blood or urine. Most preferred samples are kidney tissues.

As used herein, the term “marker” includes a polynucleotide or polypeptide molecule which is present or absent, or increased or decreased in quantity or activity in subjects following ischemia and reperfusion. Generally, the markers of the present invention are increased in quantity or activity in ischemic tissue relative to normal tissue. The relative change in quantity or activity of the marker is correlated with the degree of severity of reperfusion injury or the risk of incidence of developing severe reperfusion injury. Furthermore, as used herein, the term “therapeutic target” refers to a biochemical complex, e.g, an enzyme-substrate complex, a receptor-ligand complex or a protein-antibody complex, which is the subject of diagnostic manipulation for treating or preventing physiological injury. In the present invention, the therapeutic targets are the subject of manipulation in assays for inhibiting organ damage resulting from reperfusion, particularly in relation to organ transplantion. More specifically, the therapeutic targets of the invention include transcription factors and polynucleotides, cell surface receptors and their ligands. The present invention is particularly directed to orphan receptors where the cognate ligand has yet to be identified.

As used herein, the term “panel of markers” includes a group of markers, the quantity or activity of each member of which is correlated with the incidence or risk of incidence of reperfusion injury. In certain embodiments, a panel of markers may include only those markers that are abnormally increased in quantity or activity in subjects following reperfusion. In a preferred embodiment, the panel of markers comprises at least 5 markers, and most preferably, the panel comprises markers listed in Table 3. In other embodiments, a panel of markers may include only those markers useful for organ transplantation, such as kidney transplantation, wherein samples are taken both before and after transplantation. Various aspects of the invention are described in further detail in the following subsections:

I. Isolated Nucleic Acid Molecules

One aspect of the invention pertains to isolated nucleic acid molecules that either themselves are the genetic markers (e.g., mRNA) of the invention, or which encode the polypeptide markers of the invention, or fragments thereof. Another aspect of the invention pertains to isolated nucleic acid fragments sufficient for sue as hybridization probes to identify the nucleic acid molecules encoding the markers for the invention in a sample, as well as nucleotide fragments for use as PCR primers of the amplification or mutation of the nucleic acid molecules which encode the markers of the invention. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules that are separated from other nucleic acid molecules that are present in the natural source of the nucleic acid. For example, with regards to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated marker nucleic acid molecule of the invention, or nucleic acid molecule encoding a polypeptide marker of the invention, can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of one of the genes set forth in Tables 3-7, or a portion thereof, can be isolated using standard molecular biology techniques and the sequence information provided herein. Using all or portion of the nucleic acid sequence of one of the genes set forth in Tables 3-7 as a hybridization probe, a marker gene of the invention or a nucleic acid molecule encoding a polypeptide marker of the invention can be isolated using standard hybridization and cloning techniques (e.g., as described in Sambrook, J., Fritsch, E. F., and Maniatis, T. Molecular Cloning: A Laboratory Manual 2nd, ed., Cold Spring Harbor Laboratory, Cold Spring Harbor Laboratory Press, Cold spring Harbor, N.Y., 1989).

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to marker nucleotide sequences, or nucleotide sequences encoding a marker of the invention can be prepared by standard synthetic techniques, e.g., using an automated DNA synthesizer.

In another preferred embodiment, an isolated nucleic acid molecule of the invention comprises a nucleic acid molecule that is a complement of the nucleotide sequence of a marker of the invention (e.g., a gene set forth in Tables 3-7), or a portion of any of these nucleotide sequences. A nucleic acid molecule that is complementary to such a nucleotide sequence is one that is sufficiently complementary to the nucleotide sequence such that it can hybridize to the nucleotide sequence, thereby forming a stable duplex.

The nucleic acid molecule of the invention, moreover, may comprise only a portion of the nucleic acid sequence of a marker nucleic acid of the invention, or a gene encoding a marker polypeptide of the invention, for example, a fragment that can be used as a probe or primer. The probe/primer typically comprises substantially purified oligonucleotide. The oligonucleotide typically comprises a region of nucleotide sequence that hybridizes under stringent conditions to at least about 7 or 15, preferably about 20 or 25, more preferably about 50, 75, 100, 125, 150, 175, 200, 225, 250, 275, 300, 325, 350, 400 or more consecutive nucleotides of a marker nucleic acid, or a nucleic acid encoding a marker polypeptide of the invention.

Probes based on the nucleotide sequence of a marker gene or of a nucleic acid molecule encoding a marker polypeptide of the invention can be used to detect transcripts or genomic sequences corresponding to the marker gene(s) and/or marker polypeptide(s) of the invention. In preferred embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue(s) that misexpress (e.g., overexpress or underexpress) a marker polypeptide of the invention, or that have greater or fewer copies of a marker gene of the invention. For example, a level of a marker polypeptide-encoding nucleic acid in a sample of cells from a subject may be detected, the amount of mRNA transcript of a gene encoding a marker polypeptide may be determined, or the presence of mutations or deletions of a marker gene of the invention may be assessed.

The invention further encompasses nucleic acid molecules that differ from the nucleic acid sequences of the genes set forth in Tables 3-7 due to degeneracy of the genetic code, and thus encode the same proteins as those encoded by the genes shown in Tables 3-7.

In addition to the nucleotide sequences of the genes set forth in Tables 3-7, it will be appreciated by those skilled in the art that DNA sequence polymorphisms that lead to changes in the amino acid sequences of the proteins encoded by the genes set forth in Tables 3-7 may exist within a population, e.g., the human population). Such genetic polymorphism in the genes set forth in Tables 3-7 may exist among individuals within a population due to natural allelic variation. An allele is one of a group of genes that occur alternatively at a given genetic locus. In addition it will be appreciated that DNA polymorphisms that affect RNA expression levels can also exist that may affect the overall expression level of that gene, e.g., by affecting regulation or degradation). As used herein, the phrase “allelic variant” includes a nucleotide sequence that occurs at a given locus or a polypeptide encoded by the nucleotide sequence. As used herein, the terms “gene” and “recombinant gene” refer to nucleic acid molecules that include an open reading frame encoding a marker polypeptide of the invention.

Nucleic acid molecules corresponding to natural allelic variants and homologs of the marker genes, or genes encoding the marker proteins of the invention can be isolated based on their homology to the genes set forth in Tables 3-7, using the cDNAs disclosed herein, or a portion thereof, as a hybridization probe according to standard hybridization techniques under stringent hybridization conditions. Nucleic acid molecules corresponding to natural allelic variants and homologs of the marker genes of the invention can further be isolated by mapping to the same chromosome or locus as the marker genes or genes encoding the marker proteins of the invention.

In another embodiment, an isolated nucleic acid molecule of the invention is at least 15, 20, 25, 30, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides in length and hybridizes under stringent conditions to a nucleic acid molecule corresponding to a nucleotide sequence of a marker gene or gene encoding a marker protein of the invention. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989) 6.3.1-6.3.6. Preferably, an isolated nucleic acid molecule of the invention that hybridizes under stringent conditions to the sequence of one of the genes set forth in Tables 3-7 corresponds to a naturally occurring nucleic acid molecule. As used herein, a “naturally occurring” nucleic acid molecule includes an RNA or DNA molecule having a nucleotide sequence that occurs in nature (e.g., encodes a natural protein).

In addition to naturally occurring allelic variants of the marker gene and gene encoding a marker protein of the invention sequences that may exist in the population, the skilled artisan will further appreciate that changes can be introduced by mutation into the nucleotide sequences of the marker genes or genes encoding the marker proteins of the invention, thereby leading to changes in the amino acid sequence of the encoded proteins, without altering the functional activity of these proteins. For example, nucleotide substitutions leading to amino acid substitutions at “nonessential” amino acid residues can be made. A “nonessential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) are predicted to be particularly unamenable to alteration.

Accordingly, another aspect of the invention pertains to nucleic acid molecules encoding a marker protein of the invention that contain changes in amino acid residues that are not essential for activity. Such proteins differ in amino acid sequence from the marker proteins encoded by the genes set forth in Tables 3-7, yet retain biological activity. In one embodiment, the protein comprises an amino acid sequence at least about 60%, 65%, 70%, 75%, 80%, 85%, 90%, 95%, 98% or more homologous to a marker protein of the invention.

An isolated nucleic acid molecule encoding a protein homologous to a marker protein of the invention can be created by introducing one or more nucleotide substitutions, additions or deletions into the nucleotide sequence of the gene encoding the marker protein, such that one or more amino acid substitutions, additions or deletions are introduced into the encoded protein. Mutations can be introduced into the genes of the invention (e.g., a gene set forth in Tables 3-7) by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of a coding sequence of a gene of the invention, such as by saturation mutagenesis, and the resultant mutants can be screened for biological activity to identify mutants that retain activity. Following mutagenesis, the encoded protein can be expressed recombinantly and the activity of the protein can be determined.

Another aspect of the invention pertains to isolated nucleic acid molecules which are antisense to the marker genes and genes encoding marker proteins of the invention. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand of a gene of the invention (e.g., a gene set forth in Tables 3-7), or to only a portion thereof. In one embodiment, an antisense nucleic acid molecule is antisense to a “coding region” of the coding strand of a nucleotide sequence of the invention. The term “coding region” includes the region of the nucleotide sequence comprising codons which are translated into amino acid. In another embodiment, the antisense nucleic acid molecule is antisense to a “noncoding region” of the coding strand of a nucleotide sequence of the invention.

The term “noncoding region” includes 5′ and 3′ sequences which flank the coding region that are not translated into amino acids (i.e., also referred to as 5′ and 3′ untranslated regions).

Antisense nucleic acids of the invention can be designed according to the rules of Watson and Crick base pairing. The antisense nucleic acid molecule can be complementary to the entire coding region of an mRNA corresponding to a gene of the invention, but more preferably is an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenexine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a marker protein of the invention to thereby inhibit expression of the protein, e.g., by inhibiting transcription and/or translation. The hybridization can be by conventional nucleotide complementarity to form a stable duplex, or, for example, in the cases of an antisense nucleic acid molecule which binds to DNA duplexes, through specific interactions in the major groove of the double helix. An example of a route of administration of antisense nucleic acid molecules of the invention include direct injection at a tissue site (e.g., in kidney). Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoif and Gerlach (1988) Nature 334:585-591)) can be used to catalytically cleave mRNA transcripts of the genes of the invention (e.g., a gene set forth in Tables 3-7) to thereby inhibit translation of this mRNA. A ribozyme having specificity for a marker protein-encoding nucleic acid can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a marker protein-encoding mRNA. (See, e.g., Cech et al. U.S. Pat. No. 4,987,071; and Cech et al. U.S. Pat. No. 5,116,742). Alternatively, mRNA transcribed from a gene of the invention can be used to select a catalytic RNA having a specific ribonuclease activity from a pool of RNA molecules. See, e.g., Bartel and Szostak (1993) Science 261:1411-18.

Alternatively, expression of a gene of the invention (e.g., a gene set forth in Tables 3-7) can be inhibited by targeting nucleotide sequences complementary to the regulatory region of these genes (e.g., the promoter and/or enhancers) to form triple helical structures that prevent transcription of the gene in target cells. See generally, Helene (1991) Anticancer Drug Des. 6(6):569-84; Helene et al. (1992) Ann. N. Y. Acad Sci. 660:27-36; and Maher, L. J. (1992) Bioassays 14(12):807-15.

Expression of the marker genes, and genes encoding marker proteins of the invention, can also be inhibited using RNA interference (“RNAᵢ”). This is a technique for post transcriptional gene silencing (“PTGS”), in which target gene activity is specifically abolished with cognate double-stranded RNA (“dsRNA”). RNAᵢ resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNAᵢ in mammalian systems is disclosed in PCT application WO 00/63364 which is incorporated by reference herein in its entirety. Basically, dsRNA of at least about 600 nucleotides, homologous to the target marker is introduced into the cell and a sequence specific reduction in gene activity is observed. See generally, Ui-Tei et al. (2000) FEBS Letters 479:79-82.

In yet another embodiment, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, for example, inducing transcription or translation arrest or inhibiting replication. PNAs of the nucleic acid molecules of the invention (e.g., a gene set forth in Tables 3-7) can also be used in the analysis of single base pair mutations in a gene, (e.g., by PNA-directed PCR clamping); as ‘artificial restriction enzymes’ when used in combination with other enzymes, (e.g., S1 nucleases (Hyrup et al. (1996), supra)); or as probes or primers for DNA sequencing or hybridization (Hyrup et al. (1996), supra; Perry-O'Keefe et al. (1996), supra).

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the nucleic acid molecules of the invention can be generated that may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup et al. (1996), supra). The synthesis of PNA-DNA chimeras can be performed as described in Hyrup et al. (1996), supra and Finn et al. (1996) Nuc. Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag et al. (1989) Nuc. Acid Res. 17:5973-88). PNA monomers are then coupled in a stepwise manner to produce a chimeric molecule with a 5′ PNA segment and a 3′ DNA segment (Finn et al. (1996), supra). Alternatively, chimeric molecules can be synthesized with a 5′ DNA segment and a 3′ PNA segment (Peterser et al. (1975) Bioorganic Med Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad Sci. USA 84:648-652; PCT Publication No. W088/098 10) or the blood-kidney barrier (see, e.g., PCT Publication No. W089/10134). In addition, oligonucleotides can be modified with hybridization-triggered cleavage agents (see, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label or a fluorescent label (e.g., a molecular beacon, as described in U.S. Pat. No. 5,876,930).

II. Isolated Proteins and Antibodies

One aspect of the invention pertains to isolated marker proteins, and biologically active portions thereof, as well as polypeptide fragments suitable for use as immunogens to raise anti-marker protein antibodies. In one embodiment, native marker proteins can be isolated from cells or tissue sources by an appropriate purification scheme using standard protein purification techniques. In another embodiment, marker proteins are produced by recombinant DNA techniques. Alternative to recombinant expression, a marker protein or polypeptide can be synthesized chemically using standard peptide synthesis techniques.

An “isolated” or “purified” protein or biologically active portion thereof is substantially free of cellular material or other contaminating proteins from the cell or tissue source from which the marker protein is derived, or substantially free from chemical precursors or other chemicals when chemically synthesized. The language “substantially free of cellular material” includes preparations of marker protein in which the protein is separated from cellular components of the cells from which it is isolated or recombinantly produced. In one embodiment, the language “substantially free of cellular material” includes preparations of marker protein having less than about 30% (by dry weight) of nonmarker protein (also referred to herein as a “contaminating protein”), more preferably less than about 20% of nonmarker protein, still more preferably less than about 10% of nonmarker protein, and most preferably less than about 5% nonmarker protein. When the marker protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of marker protein in which the protein is separated from chemical precursors or other chemicals which are involved in the synthesis of the protein. In one embodiment, the language “substantially free of chemical precursors or other chemicals” includes preparations of protein having less than about 30% (by dry weight) of chemical precursors or nonprotein chemicals, more preferably less than about 20% chemical precursors or nonprotein chemicals, still more preferably less than about 10% chemical precursors or nonprotein chemicals, and most preferably less than about 5% chemical precursors or nonprotein chemicals.

As used herein, a “biologically active portion” of a marker protein includes a fragment of a marker protein comprising amino acid sequences sufficiently homologous to or derived from the amino acid sequence of the marker protein, which include fewer amino acids than the full length marker proteins, and exhibit at least one activity of a marker protein. Typically, biologically active portions comprise a domain or motif with at least one activity of the marker protein. A biologically active portion of a marker protein can be a polypeptide that is, for example, 10, 25, 50, 100, 200 or more amino acids in length. Biologically active portions of a marker protein can be used as targets for developing agents which modulate a marker protein-mediated activity.

In a preferred embodiment, marker protein is encoded by a gene set forth in Tables 3-7. In other embodiments, the marker protein is substantially homologous to a marker protein encoded by a gene set forth in Tables 3-7, and retains the functional activity of the marker protein, yet differs in amino acid sequence due to natural allelic variation or mutagenesis, as described in detail in subsection I above. Accordingly, in another embodiment, the marker protein is a protein which comprises an amino acid sequence at least about 60%, 65%, 70%, 75%, 80%, 85%, 90%, 95%, 98% or more homologous to the amino acid sequence encoded by a gene set forth in Tables 3-7.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and nonhomologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. (48):444-453) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller ((1989) CABIOS 4:11-17) which has been incorporated into the ALIGN program (version 2.0), using a PAM 120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to marker protein molecules of the invention. To obtain gapped alignments for comparison purposes, Gapped BLAST can be utilized as described in Altschul et al. (1997) Nuc. Acids Res. 25(17):3389-3402. When utilizing BLAST and Gapped BLAST programs, the default parameters of the respective programs (e.g., XBLAST and NBLAST) can be used. See http://www.ncbi.nim.nih.gov.

The invention also provides chimeric or fusion marker proteins. As used herein, a marker “chimeric protein” or “fusion protein” comprises a marker polypeptide operatively linked to a nonmarker polypeptide. A “marker polypeptide” includes a polypeptide having an amino acid sequence encoded by a gene set forth in Tables 3-7, whereas a “nonmarker polypeptide” includes a polypeptide having an amino acid sequence corresponding to a protein that is not substantially homologous to the marker protein, e.g., a protein that is different from the marker protein and that is derived from the same or a different organism. Within a marker fusion protein the polypeptide can correspond to all or a portion of a marker protein. In a preferred embodiment, a marker fusion protein comprises at least one biologically active portion of a marker protein. Within the fusion protein, the term “operatively linked” is intended to indicate that the marker polypeptide and the nonmarker polypeptide are fused in-frame to each other. The nonmarker polypeptide can be fused to the N-terminus or C-terminus of the marker polypeptide.

For example, in one embodiment, the fusion protein is a GST-marker fusion protein in which the marker sequences are fused to the C-terminus of the GST sequences. Such fusion proteins can facilitate the purification of recombinant marker proteins.

In another embodiment, the fusion protein is a marker protein containing a heterologous signal sequence at its N-terminus. In certain host cells (e.g., mammalian host cells), expression and/or secretion of marker proteins can be increased through use of a heterologous signal sequence. Such signal sequences are well known in the art.

The marker fusion proteins of the invention can be incorporated into pharmaceutical compositions and administered to a subject in vivo, as described herein. The marker fusion proteins can be used to affect the bioavailability of a marker protein substrate. Use of marker fusion proteins may be useful therapeutically for the treatment of, or prevention of damage (e.g., organ damage resulting from reperfusion) caused by, for example, (i) aberrant modification or mutation of a gene encoding a marker protein; (ii) misregulation of the marker protein-encoding gene; and (iii) aberrant posttranslational modification of a marker protein.

Moreover, the marker-fusion proteins of the invention can be used as immunogens to produce anti-marker protein antibodies in a subject, to purify marker protein ligands, and in screening assays to identify molecules that inhibit the interaction of a marker protein with a marker protein substrate.

Preferably, a marker chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Current Protocols In Molecular Biology, eds. Ausubel et al. John Wiley & Sons: 1992). Moreover, many expression vectors are commercially available that already encode a fusion moiety (e.g., a GST polypeptide). A marker protein-encoding nucleic acid can be cloned into such an expression vector such that the fusion moiety is linked in-frame to the marker protein.

A signal sequence can be used to facilitate secretion and isolation of the secreted protein or other proteins of interest. Signal sequences are typically characterized by a core of hydrophobic amino acids that are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein that is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

Alternatively, the signal sequence can be linked to the protein of interest using a sequence that facilitates purification, such as with a GST domain.

The present invention also pertains to variants of the marker proteins of the invention which function as either agonists (mimetics) or as antagonists to the marker proteins. Variants of the marker proteins can be generated by mutagenesis, e.g., discrete point mutation or truncation of a marker protein. An agonist of the marker proteins can retain substantially the same, or a subset, of the biological activities of the naturally occurring form of a marker protein. An antagonist of a marker protein can inhibit one or more of the activities of the naturally occurring form of the marker protein by, for example, competitively modulating an activity of a marker protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the marker protein.

Variants of a marker protein that function as either marker protein agonists (mimetics) or as marker protein antagonists can be identified by screening combinatorial libraries of mutants, e.g., truncation mutants, of a marker protein for marker protein agonist or antagonist activity. In one embodiment, a variegated library of marker protein variants is generated by combinatorial mutagenesis at the nucleic acid level and is encoded by a variegated gene library. A variegated library of marker protein variants can be produced by, for example, enzymatically ligating a mixture of synthetic oligonucleotides into gene sequences such that a degenerate set of potential marker protein sequences is expressible as individual polypeptides, or alternatively, as a set of larger fusion proteins (e.g., for phage display) containing the set of marker protein sequences therein. There are a variety of methods which can be used to produce libraries of potential marker protein variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential marker protein sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1055; Ike et al. (1983) Nuc. Acid Res. 11:477).

In addition, libraries of fragments of a protein coding sequence corresponding to a marker protein of the invention can be used to generate a variegated population of marker protein fragments for screening and subsequent selection of variants of a marker protein. In one embodiment, a library of coding sequence fragments can be generated by treating a double stranded PCR fragment of a marker protein coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived that encodes N-terminal, C-terminal and internal fragments of various sizes of the marker protein.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify marker variants (Arkin and Yourvan (1992) Proc. Natl. Acad. Sci. USA 89:7811-7815; Delgrave et al. (1993) Protein Engineering 6(3):327-331).

An isolated marker protein, or a portion or fragment thereof, can be used as an immunogen to generate antibodies that bind marker proteins using standard techniques for polyclonal and monoclonal antibody preparation. A full-length marker protein can be used or, alternatively, the invention provides antigenic peptide fragments of these proteins for use as immunogens. The antigenic peptide of a marker protein comprises at least 8 amino acid residues of an amino acid sequence encoded by a gene set forth in Tables 3-7, and encompasses an epitope of a marker protein such that an antibody raised against the peptide forms a specific immune complex with the marker protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of the marker protein that are located on the surface of the protein, e.g., hydrophilic regions, as well as regions with high antigenicity.

A marker protein immunogen typically is used to prepare antibodies by immunizing a suitable subject, (e.g., rabbit, goat, mouse or other mammal) with the immunogen. An appropriate immunogenic preparation can contain, for example, recombinantly expressed marker protein or a chemically synthesized marker polypeptide. The preparation can further include an adjuvant, such as Freund's complete or incomplete adjuvant, or similar immunostimulatory agent. Immunization of a suitable subject with an immunogenic marker protein preparation induces a polyclonal anti-marker protein antibody response.

Accordingly, another aspect of the invention pertains to anti-marker protein antibodies. The term “antibody” as used herein includes immunoglobulin molecules and immunologically active portions of immunoglobulin molecules, i.e., molecules that contain an antigen binding site that specifically binds (immunoreacts with) an antigen, such as a marker protein. Examples of immunologically active portions of immunoglobulin molecules include F(ab) and F(ab′)₂ fragments that can be generated by treating the antibody with an enzyme such as pepsin. The invention provides polyclonal and monoclonal antibodies that bind to marker proteins. The term “monoclonal antibody” or “monoclonal antibody composition”, as used herein, includes a population of antibody molecules that contain only one species of an antigen binding site capable of immunoreacting with a particular epitope. A monoclonal antibody composition thus typically displays a single binding affinity for a particular marker protein with which it immunoreacts.

Polyclonal anti-marker protein antibodies can be prepared as described above by immunizing a suitable subject with a marker protein of the invention. The anti-marker protein antibody titer in the immunized subject can be monitored over time by standard techniques, such as with an enzyme linked immunosorbent assay (ELISA) using immobilized marker protein. If desired, the antibody molecules directed against marker proteins can be isolated from the mammal (e.g., from the blood) and further purified by well known techniques, such as protein A chromatography, to obtain the IgG fraction. At an appropriate time after immunization, e.g., when the anti-marker protein antibody titers are highest, antibody-producing cells can be obtained from the subject and used to prepare monoclonal antibodies by standard techniques, such as the hybridoma technique originally described by Kohler and Milstein (1975) Nature 256:495-497 (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-3I; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96), or trioma techniques. The technology for producing monoclonal antibody hybridomas is well known (see generally, R. H. Kenneth, in Monoclonal Antibodies: A New Dimension In Biological Analyses, Plenum Publishing Corp., New York, N.Y. (1980); E. A. Lerner (1981) Yale J. Biol. Med. 54:387-402; Gefter et al. (1977) Somatic Cell Genet. 3:231-36). Briefly, an immortal cell line (typically a myeloma) is fused to lymphocytes (typically splenocytes) from a mammal immunized with a marker protein immunogen as described above, and the culture supernatants of the resulting hybridoma cells are screened to identify a hybridoma producing a monoclonal antibody that binds to a marker protein of the invention.

Any of the many well known protocols used for fusing lymphocytes and immortalized cell lines can be applied for the purpose of generating an anti-marker protein monoclonal antibody (see, e.g., Galfre et al. (1977) Nature 266:SSOS2; Gefter et al. Somatic Cell Genet., cited supra; Letter, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, axninopterin and thymidine (“HAT medium”). Any of a number of myeloma cell lines can be used as a fusion partner according to standard techniques, e.g., the P3-NS1/1-Ag4-1, P3-x63-Ag8.653 or Sp210-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind to a marker protein, e.g., using a standard ELISA assay.

As an alternative to preparing monoclonal antibody-secreting hybridomas, a monoclonal anti-marker protein antibody can be identified and isolated by screening a recombinant combinatorial immunoglobulin library (e.g., an antibody phage display library) with marker protein to thereby isolate immunoglobulin library members that bind to a marker protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92115679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-marker protein antibodies, such as chimeric and humanized monoclonal antibodies, comprising both human and nonhuman portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad Sci. USA 84:3439-3443; Liu et al. (1987) J. Immunol. 139:3521 3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Humanized antibodies are particularly desirable for therapeutic treatment of human subjects. Humanized forms of nonhuman (e.g. murine) antibodies are chimeric molecules of immunoglobulins, immunoglobulin chains or fragments thereof (such as Fv, Fab, Fab′, F(ab′)₂ or other antigen-binding subsequences of antibodies) that contain minimal sequence derived from nonhuman immunoglobulin. Humanized antibodies include human immunoglobulins (recipient antibody) in which residues forming a complementary determining region (CDR) of the recipient are replaced by residues from a CDR of a nonhuman species (donor antibody) such as mouse, rat or rabbit having the desired specificity, affinity and capacity. In some instances, Fv framework residues of the human immunoglobulin are replaced by corresponding nonhuman residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a nonhuman immunoglobulin and all or substantially all of the constant regions being those of a human immunoglobulin consensus sequence. The humanized antibody will preferably also comprise at least a portion of an immunoglobulin constant region (Fc), typically that of a human immunoglobulin (Jones et al. (1986) Nature 321:522-525; Riechmann et al. (1988) Nature 323:323-329; and Presta (1992) Curr. Op. Struct. Biol. 2:594-596).

Such humanized antibodies can be produced using transgenic mice that are incapable of expressing endogenous immunoglobulin heavy and light chain genes, but that can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing humanized antibodies, see Lonberg and Huszar (1995) Int. Rev. Immunol. 13:65-93). For a detailed discussion of this technology for producing humanized antibodies and humanized monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide humanized antibodies directed against a selected antigen using technology similar to that described above.

Humanized antibodies which recognize a selected epitope can be generated using a technique referred to as “guided selection.” In this approach a selected nonhuman monoclonal antibody, e.g., a murine antibody, is used to guide the selection of a humanized antibody recognizing the same epitope (Jespers et al. (1994) Bio/technology 12:899-903).

An anti-marker protein antibody (e.g., monoclonal antibody) can be used to isolate a marker protein of the invention by standard techniques, such as affinity chromatography or immunoprecipitation. An anti-marker protein antibody can facilitate the purification of natural marker proteins from cells and of recombinantly produced marker proteins expressed in host cells. Moreover, an anti-marker protein antibody can be used to detect marker protein (e.g., in a cellular lysate or cell supernatant) in order to evaluate the abundance and pattern of expression of the marker protein. Anti-marker protein antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a marker protein of the invention (or a portion thereof). As used herein, the term “vector” includes a nucleic acid molecule capable of transporting another nucleic acid to which it has been linked. One type of vector is a “plasmid”, which includes a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably, as the plasmid is the most commonly used form of vector. However, the invention is intended to include other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequences) in a manner that allows for expression of the nucleotide sequence (e.g., in an in vitro transcription/translation system or in a host cell if the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., marker proteins, mutant forms of marker proteins, fusion proteins, and the like).

The recombinant expression vectors of the invention can be designed for expression of marker proteins in prokaryotic or eukaryotic cells. For example, marker proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or nonfusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRITS (Pharmacia, Piscataway, N.J.) which fuse glutathione S transferase (GST), maltose E binding protein, or protein A, respectively, to the target recombinant protein.

Purified fusion proteins can be utilized in marker activity assays, (e.g., direct assays or competitive assays described in detail below), or to generate antibodies specific for marker proteins, for example.

Examples of suitable inducible nonfusion E. coli expression vectors include pTrc (Amann et al. (1988) Gene 69:301-315) and pET 11d (Studier et al. Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wade et al. (1992) Nucleic Acids Res. 20:2111-2118). Such alteration of nucleic acid sequences of the invention can be carried out by standard DNA synthesis techniques.

In another embodiment, the marker protein expression vector is a yeast expression vector. Examples of vectors for expression in yeast S. cerevisiae include pYepSec1(Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and picZ (Invitrogen Corp, San Diego, Calif.).

Alternatively, marker proteins of the invention can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-I95). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsch, E. F., and Maniatis, T. Molecular Cloning: A Laboratory Manual. 2^{nd}, ed. Cold Spring Expression Technology: Methods in Enzymology 185, Academic Press, San Diego, Calif. (1990) 60-89. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the marker protein expression vector is a yeast expression vector. Examples of vectors for expression in yeast S. cerevisiae include pYepSec1 (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pIC (InVitrogen Corp, San Diego, Calif.).

Alternatively, marker proteins of the invention can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsch, E. F., and Maniatis, T. Molecular Cloning: A Laboratory Manual. 2^{nd}, ed, Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Nonlimiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter, Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter, U.S. Pat. No. 4,873,316 and European Application Publication No. 264,166). Developmentally regulated promoters are also encompassed, for example the marine hox promoters (Kessel and Grass (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule which is antisense to mRNA corresponding to a gene of the invention (e.g., a gene set forth in Tables 3-7). Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (1986) Antisense RNA as a molecular tool for genetic analysis, Reviews—Trends in Genetics, Vol. 1(1).

Another aspect of the invention pertains to host cells into which a nucleic acid molecule of the invention is introduced, e.g., a gene set forth in Tables 3-7 within a recombinant expression vector or a nucleic acid molecule of the invention containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a marker protein of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DAKD-dextran-mediated transfection, lipofection, or electroporation. Suitable methods for transforming or transferring host cells can be found in Sambrook et al. (Molecular Cloning: A Laboratory Manual. 2^{nd}, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Nucleic acid encoding a selectable flag can be introduced into a host cell on the same vector as that encoding a marker protein or can be introduced on a separate vector. Cells stably transfected with the introduced nucleic acid can be identified by drug selection (e.g., cells that have incorporated the selectable flag gene will survive, while the other cells die).

A host cell of the invention, such as a prokaryotic or eukaryotic host cell in culture, can be used to produce (i.e., express) a marker protein. Accordingly, the invention further provides methods for producing a marker protein using the host cells of the invention. In one embodiment, the method comprises culturing the host cell of invention (into which a recombinant expression vector encoding a marker protein has been introduced) in a suitable medium such that a marker protein of the invention is produced. In another embodiment, the method further comprises isolating a marker protein from the medium or the host cell.

The host cells of the invention can also be used to produce nonhuman transgenic animals. For example, in one embodiment, a host cell of the invention is a fertilized oocyte or an embryonic stem cell into which marker-protein-coding sequences have been introduced. Such host cells can then be used to create nonhuman transgenic animals in which exogenous sequences encoding a marker protein of the invention have been introduced into their genome or homologous recombinant animals in which endogenous sequences encoding the marker proteins of the invention have been altered. Such animals are useful for studying the function and/or activity of a marker protein and for identifying and/or evaluating modulators of marker protein activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, and the tike. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous gene of the invention (e.g., a gene set forth in Tables 3-7) has been altered by homologous recombination between the endogenous gene and an exogenous DNA molecule introduced into a cell of the animal, e.g., an embryonic cell of the animal, prior to development of the animal.

A transgenic animal of the invention can be created by introducing a marker-encoding nucleic acid into the mate pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of a marker protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al.; U.S. Pat. No. 4,873,191 by Wagner et al.; and Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying a transgene encoding a marker protein can further be bred to other transgenic animals carrying other transgenes.

To create a homologous recombinant animal, a vector is prepared that contains at least a portion of a gene of the invention into which a deletion, addition or substitution has been introduced to thereby alter, e.g., functionally disrupt, the gene. The gene can be a human gene, but more preferably, is a nonhuman homologue of a human gene of the invention (e.g., a gene set forth in Tables 3-7). For example, a mouse gene can be used to construct a homologous recombination nucleic acid molecule, e.g., a vector, suitable far altering an endogenous gene of the invention in the mouse genome. In a preferred embodiment, the homologous recombination nucleic acid molecule is designed such that, upon homologous recombination, the endogenous gene of the invention is functionally disrupted (i.e., no longer encodes a functional protein; also referred to as a “knock out” vector). Alternatively, the homologous recombination nucleic acid molecule can be designed such that, upon homologous recombination, the endogenous gene is mutated or otherwise altered but still encodes functional protein (e.g., the upstream regulatory region can be altered to thereby alter the expression of the endogenous marker protein). In the homologous recombination nucleic acid molecule, the altered portion of the gene of the invention is flanked at its 5′ and 3′ ends by additional nucleic acid sequence of the gene of the invention to allow for homologous recombination to occur between the exogenous gene carried by the homologous recombination nucleic acid molecule and an endogenous gene in a cell, e.g., an embryonic stem cell. The additional flanking nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced gene has homologously recombined with the endogenous gene are selected (see, e.g., Li et al. (1992) Cell 69:915). The selected cells can then be injected into a blastocyst of an animal (e.g., a mouse) to form aggregation chimeras (see, e.g., Bradley, S A. in Teratocareirtomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic nonhuman animals can be produced which contain selected systems which allow for regulated expression of the transgene. One example of such a system is the cre/loxP recombinase system of bacteriophage P1. For a description of the cre/loxP recombinase system, see, e.g., Laksa et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The nucleic acid molecules of the invention (e.g., the genes set forth in Tables 3-7), fragments of marker proteins, and anti-marker protein antibodies of the invention can be incorporated into pharmaceutical compositions suitable for administration. Such compositions (also referred to herein as “bioactive agents”) typically comprise the nucleic acid molecule, protein, or antibody and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary bioactive agents can also be incorporated into the compositions.

The invention includes methods for preparing pharmaceutical compositions for modulating the expression or activity of a polypeptide or nucleic acid corresponding to a marker of the invention. Such methods comprise formulating a pharmaceutically acceptable carrier with an agent that modulates expression or activity of a polypeptide or nucleic acid corresponding to a marker of the invention. Such compositions can further include additional active agents. Thus, the invention further includes methods for preparing a pharmaceutical composition by formulating a pharmaceutically acceptable carrier with an agent that modulates expression or activity of a polypeptide or nucleic acid corresponding to a marker of the invention and one or more additional bioactive agents.

The invention also provides methods (also referred to herein as “screening assays”) for identifying modulators, i.e., candidate or test compounds or agents comprising therapeutic moieties (e.g., peptides, peptidomimetics, peptoids, small molecules or other drugs) that (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker.

The test compounds of the present invention may be either small molecules or bioactive agents. In one preferred embodiment the test compound is a small molecule. In another preferred embodiment, the test compound is a bioactive agent. Bioactive agents include naturally occurring compounds or molecules (“biomolecules”) having bioactivity in mammals, as well as proteins, peptides, oligopeptides, polysaccharides, nucleotides and polynucleotides. Preferably, the bioactive agent is a protein, polynucleotide or biomolecule. One skilled in the art will appreciate that the nature of the test compound may vary depending on the nature of the protein encoded by the marker of the invention. For example, if the marker encodes an orphan receptor having an unkown ligand, the test compound may be any of a number of bioactive agents which may act as cognate ligand, including but not limited to, cytokines, lipid-derived mediators, small biogenic amines, hormones, neuropeptides, or proteases.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, nonpeptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, nonpeptide oligomer or small molecule libraries of compounds (Lam (1997) Anticancer Drug Des. 12:145).

As used herein, the term “specific factor” refers to a bioactive agent which serves as either a substrate for a protein encoded by a marker of the invention, or alternatively, as a ligand having binding affinity to the protein. As mentioned above, the bioactive agent may be any of a variety of naturally occurring compounds, biomolecules, proteins, peptides, oligopeptides, polysaccharides, nucleotides or polynucleotides.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The earner can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a marker protein or an anti-marker protein antibody) in the required amount in an appropriate solvent with one or a combination of ingredients enmnerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Computer Readable Means and Arrays

Computer readable media comprising a marker(s) of the present invention is also provided. As used herein, “computer readable media” includes a medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a marker of the present invention.

As used herein, “recorded” includes a process for storing information on computer readable medium. Those skilled in the art can readily adopt any of the presently known methods for recording information on computer readable medium to generate manufactures comprising the markers of the present invention.

A variety of data processor programs and formats can be used to store the marker information of the present invention on computer readable medium. For example, the nucleic acid sequence corresponding to the markers can be represented in a word processing text file, formatted in commercially available software such as WordPerfect and Microsoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the markers of the present invention.

By providing the markers of the invention in computer readable form, one can routinely access the marker sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

The invention also includes an array comprising a marker(s) of the present invention, i.e., a biochip. The array can be used to assay expression of one or more genes in the array. In one embodiment, the array can be used to assay gene expression in a tissue to ascertain tissue specificity of genes in the array. In this manner, up to about 8600 genes can be simultaneously assayed for expression. This allows an expression profile to be developed showing a battery of genes specifically expressed in one or more tissues at a given point in time.

In addition to such qualitative determination, the invention allows the quantitation of gene expression in the biochip. Thus, not only tissue specificity, but also the level of expression of a battery of genes in the tissue is ascertainable. Genes can be grouped on the basis of their tissue expression per se and level of expression in that tissue. As used herein, a “normal level of expression” refers to the level of expression of a gene provided in a control sample, typically the control is taken from taken either pre-reperfusion or from a subject who has not suffered the causative event resulting in reperfusion. Furthermore, as used herein, a “normalized” expression level occurs if the expression level of an otherwise ischemic sample is rendered the same or similar to a control sample by having an expression level within the normal standard deviation for a control sample. The determination of normal levels of expression is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the arrays can be used to monitor the time course of expression of one or more genes in the array. This can occur in various biological contexts, as disclosed herein, for example development and differentiation, disease progression, in vitro processes, such a cellular transformation and senescence, autonomic neural and neurological processes, such as, for example, pain and appetite, and cognitive functions, such as learning or memory.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal versus ischemic cells. This provides a battery of genes that could serve as a molecular target for diagnosis or therapeutic intervention. In particular, biochips can be made comprising arrays not only of the differentially expressed markers listed in Tables 3-7, but of markers specific to subjects suffering from specific manifestations or degrees of the disease (i.e., facial lesions, nephritis, endocarditis, hemolytic anemia and leukopenia).

VI. Predictive Medicine

The present invention pertains to the field of predictive medicine in which diagnostic assays, prognostic assays, pharmacogenetics and monitoring clinical trials are used for prognostic (predictive) purposes to thereby treat an individual prophylactically. Accordingly, one aspect of the present invention relates to diagnostic assays for determining marker protein and/or nucleic acid expression as welt as marker protein activity, in the context of a biological sample (e.g., blood, serum, cells, tissue) to thereby determine whether an individual is at risk for developing organ damage resulting from reperfusion associated with increased marker protein expression or activity. The invention also provides for prognostic (or predictive) assays for determining whether an individual is at risk of developing organ damage associated with marker protein, nucleic acid expression or activity. For example, the number of copies of a marker gene can be assayed in a biological sample. Such assays can be used for prognostic or predictive purposes to thereby phophylactically treat an individual prior to the onset of permanent organ damage (or acute rejection in transplants), characterized by or associated with marker protein, nucleic acid expression or activity.

Another aspect of the invention pertains to monitoring the influence of agents (e.g., drugs, compounds) on the expression or activity of marker in clinical trials.

These and other agents are described in further detail in the following sections.

1. High-Throughput Screening Assays

Recent advancements have provided a number of methods to detect binding activity between bioactive agents. Common methods of high-throughput screening involve the use of of fluorescence-based technology, including but not limited, to BRET® or FRET® (both by Packard Instrument Co., Meriden, Conn.) which measure the detection signal provided by the proximity of bound fluorophores. By combining test compounds with proteins encoded by the markers of the invention and determining the binding activity between such, diagnostic analysis can be performed to elucidate the coupling systems. Generic assays using cytosensor microphysiometer may also be used to measure metabolic activation, while changes in calcium mobilization can be detected by using the fluorescence-based techniques such as FLIPR® (Molecular Devices Corp, Sunnyvale, Calif.). In addition, the presence of apoptotic cells may be determined by TUNEL assay, which utilizes flow cytometry to detect free 3-OH termini resulting from cleavage of genomic DNA during apoptosis. As mentioned above, a variety of functional assays well-known in the art may be used in combination to screen and/or study the reactivity of different types of activating test compounds. Preferably, the high-throughput screening assay of the present invention utilizes label-free plasmon resonance technology as provided by BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and nucleic acids), and since the BIACORE® sensor surface can be functionalized to bind a variety of these bioactive agents, detection of a wide selection of test compounds can thus be accomplished.

Therefore, the invention provides for high-throughput screening of test compounds for the ability to inhibit activity of a protein encoded by the markers listed in Tables 3-7 by combining the test compounds and the protein in high-throughput assays such as BIACORE®, or in fluorescence based assays such as BRET®. In addition, high-throughput assays may be utilized to identify specific factors that bind to the encoded proteins, or alternatively, to identify test compounds that prevent binding of the receptor to the specific factor. In the case of orphan receptors, the specific factor may be the natural ligand for the receptor. Moreover, the high-throughput screening assays may be modified to determine whether test compounds can bind to either the encoded protein or to the specific factor (e.g., substrate or ligand) that binds to the protein.

2. Diagnostic Assays

An exemplary method for detecting the presence or absence of marker protein or nucleic acid of the invention in a biological sample involves obtaining a biological sample from a test subject and contacting the biological sample with a compound or an agent capable of detecting the protein or nucleic acid (e.g., mRNA, genomic DNA) that encodes the marker protein such that the presence of the marker protein or nucleic acid is detected in the biological sample. A preferred agent for detecting mRNA or genomic DNA corresponding to a marker gene or protein of the invention is a labeled nucleic acid probe capable of hybridizing to a mRNA or genomic DNA of the invention. Suitable probes for use in the diagnostic assays of the invention are described herein.

A preferred agent for detecting marker protein is an antibody capable of binding to a marker protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to defeat marker mRNA, protein, or genomic DNA in a biological sample in vitro as well as in vivo. For example, in vitro techniques for detection of marker mRNA include Northern hybridizations and in situ hybridizations. In vitro techniques for detection of marker protein include enzyme linked immunosorbent assays (ELISAs), Western blots, immunoprecipitations and immunofluoresCence. In vitro techniques for detection of marker genomic DNA include Southern hybridizations. Furthermore, in vivo techniques for detection of marker protein include introducing into a subject a labeled anti-marker antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a subject, contacting the control sample with a compound or agent capable of detecting marker protein, mRNA, or genomic DNA, such that the presence of marker protein, mRNA or genomic DNA is detected in the biological sample, and comparing the presence of marker protein, mRNA or genomic DNA in the control sample with the presence of marker protein, mRNA or genomic DNA in the test sample.

The invention also encompasses kits for detecting the presence of marker in a biological sample. For example, the kit can comprise a labeled compound or agent capable of detecting marker protein or mRNA in a biological sample; means for determining the amount of marker in the sample; and means for comparing the amount of marker in the sample with a standard. The compound or agent can be packaged in a suitable container. The kit can further comprise instructions for using the kit to detect marker protein or nucleic acid.

3. Prognostic Assays

The diagnostic methods, described herein can furthermore be utilized to identify subjects having or at risk of developing organ damage from reperfusion associated with aberrant marker expression or activity. As used herein, the term “aberrant” includes a marker expression or activity that deviates from the wild type marker expression or activity. Aberrant expression or activity includes increased or decreased expression or activity, as well as expression or activity that does not follow the wild type developmental pattern of expression or the subcellular pattern of expression. For example, aberrant marker expression or activity is intended to include the cases in which a mutation in the marker gene causes the marker gene to be under-expressed or over-expressed and situations in which such mutations result in a nonfunctional marker protein or a protein that does not function in a wild-type fashion, e.g., a protein that does not interact with a marker ligand or one that interacts with a nonmarker protein ligand. In the present invention, as related to ischemia and reperfusion injury, aberrant expression or activity is typically correlated with an abnormal increase.

The assays described herein, such as the preceding diagnostic assays or the following assays, can be utilized to identify a subject having or at risk of developing organ damage associated with a misregulation in marker protein activity or nucleic acid expression following reperfusion. Alternatively, the prognostic assays can be utilized to identify a subject having or at risk for developing organ damage associated with a misregulation in marker protein activity or nucleic acid expression following reperfusion. Thus, the present invention provides a method for identifying severe reperfusion injury associated with aberrant marker expression or activity in which a test sample is obtained from a subject and marker protein or nucleic acid (e.g., mRNA or genomic DNA) is detected, wherein the presence of marker protein or nucleic acid is diagnostic for a subject having or at risk of developing organ damage associated with aberrant marker expression or activity. As used herein, a “test sample” includes a biological sample obtained from a subject of interest. For example, a test sample can be a biological fluid (e.g., blood PBMCs), cell sample, or tissue (e.g., kidney).

Furthermore, the prognostic assays described herein can be used to determine whether a subject can be administered an agent (e.g., an agonist, antagonist, peptidomimetic, protein, peptide, nucleic acid, small molecule, or other drug candidate) to treat or prevent organ damage from reperfusion as associated with increased marker expression or activity. For example, such methods can be used to determine whether a subject can be effectively treated with an agent to inhibit organ damage resulting from reperfusion. Thus, the present invention provides methods for determining whether a subject can be effectively treated with an agent for an injury associated with increased marker expression or activity in which a test sample is obtained and marker protein or nucleic acid expression or activity is detected (e.g., wherein the abundance of marker protein or nucleic acid expression or activity is diagnostic for a subject that can be administered the agent to treat an injury associated with increased marker expression or activity).

In relation to the field of organ transplantation, prognostic assays can be devised to determine whether a subject undergoing organ transplantation has a poor outlook for long term organ survival, as provided by determining the potential for acute rejection. In a preferred embodiment, prognosis can be determined shortly after transplantion, within a few days. By establishing expression profiles of different stages of reperfusion injury, from onset to acute rejection, an expression pattern may emerge to correlate a particular expression profile to increased likelihood of acute of rejection. The prognosis may then be used to devise a more aggressive treatment program to avert chronic rejection of the organ and ensure long-term survival.

The methods of the invention can also be used to detect genetic alterations in a marker gene, thereby determining if a subject with the altered gene is at risk for damage characterized by misregulation in marker protein activity or nucleic acid expression, such as ischemia or reperfusion injury. In preferred embodiments, the methods include detecting, in a sample of cells from the subject, the presence or absence of a genetic alteration characterized by at least one of an alteration affecting the integrity of a gene encoding a marker-protein, or the mis-expression of the marker gene. For example, such genetic alterations can be detected by ascertaining the existence of at least one of 1) a deletion of one or more nucleotides from a marker gene; 2) an addition of one or more nucleotides to a marker gene; 3) a substitution of one or more nucleotides of a marker gene, 4) a chromosomal rearrangement of a marker gene; 5) an alteration in the level of a messenger RNA transcript of a marker gene, 6) aberrant modification of a marker gene, such as of the methylation pattern of the genomic DNA, 7) the presence of a non-wild type splicing pattern of a messenger RNA transcript of a marker gene, 8) a non-wild type level of a marker-protein, 9) allelic loss of a marker gene, and 10) inappropriate post-translational modification of a marker-protein. As described herein, there are a large number of assays known in the art that can be used for detecting alterations in a marker gene. A preferred biological sample is a tissue (e.g., kidney) or blood sample isolated by conventional means from a subject.

In certain embodiments, detection of the alteration involves the use of a probe/primer in a polymerase chain reaction (PCR) (see, e.g., U.S. Pat. Nos. 4,683,995 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the marker-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers that specifically hybridize to a marker gene under conditions such that hybridization and amplification of the marker-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a marker gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in a marker gene or a gene encoding a marker protein of the invention can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations in marker can be identified in two dimensional arrays containing light generated DNA probes as described in Cronin, et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild type gene and the other complementary to the mutant gene.

In yet another embodiment, any of a variety of sequencing reactions known in the art can be used to directly sequence the marker gene and detect mutations by comparing the sequence of the sample marker with the corresponding wild type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94116101; Cohen et al. (1996) Adv. Chromatogr. 36:127-162; and Griffin et al. (1993) Appl. Biochem. Biotechnol. 38:147-159).

Other methods for detecting mutations in the marker gene or gene encoding a marker protein of the invention include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes by hybridizing (labeled) RNA or DNA containing the wild type marker sequence with potentially mutant RNA or DNA obtained from a tissue sample. The double-stranded duplexes are treated with an agent which cleaves single-stranded regions of the duplex that exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 517:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1652). According to an exemplary embodiment, a probe based on a marker sequence, e.g., a wild type marker sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in marker genes or genes encoding a marker protein of the invention. For example, single strand conformation polymorphism (SSCP) may be used to detect differences in electrophoretic mobility between mutant and wild type nucleic acids (Orita et al. (1989) Proc Natl. Acad. Sci USA:86:2766; see also Cotton (1993) Mutat. Res. 285:125-144; and Hayashi (1992) Genet. Anal. Tech Appl. 9:73-79). Single-stranded DNA fragments of sample and control marker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in elecrtophoretic mobility (Keen et al. (1991) Trends Genet. 7:5).

In yet another embodiment the movement of mutant or wild type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnose subjects exhibiting symptoms or family history of a disease or illness involving a marker gene.

Furthermore, any cell type or tissue in which marker is expressed may be utilized in the prognostic assays described herein.

4. Monitoring of Effects During Clinical Trials

Monitoring the influence of agents (e.g., drugs) on the expression or activity of a marker protein (e.g., the modulation of genes involved in ischemia and reperfusion injury) can be applied not only in basic drug screening, but also in clinical trials. For example, the effectiveness of an agent determined by a screening assay as described herein to decrease marker gene expression, protein levels, or downregulate marker activity, can be monitored in clinical trials of subjects exhibiting increased marker gene expression, protein levels, or upregulated marker activity. In such clinical trials, the expression or activity of a marker gene, and preferably, other genes that have been implicated in, for example, marker-associated damage (e.g., resulting from ischemia or reperfusion) can be used as a “read out” or markers of the phenotype of a particular cell.

For example, and not by way of limitation, genes, including marker genes and genes encoding a marker protein of the invention, that are modulated in cells by treatment with an agent (e.g., compound, drug or small molecule) that modulates marker activity (e.g., identified in a screening assay as described herein) can be identified. Thus, to study the effect of agents on marker-associated damage (e.g., resulting from ischemia and reperfusion), for example, in a clinical trial, cells can be isolated and RNA prepared and analyzed for the levels of expression of marker and other genes implicated in the marker-associated damage, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of marker or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of: (i) obtaining a pre-administration sample from a subject prior to administration of the agent; (ii) detecting the level of expression of a marker protein, mRNA, or genomic DNA in the pre-administration sample; (iii) obtaining one or more post-administration samples from the subject; (iv) detecting the level of expression or activity of the marker protein, mRNA, or genomic DNA in the post-administration samples; (v) comparing the level of expression or activity of the marker protein, mRNA, or genomic DNA in the pre-administration sample with the marker protein, mRNA, or genomic DNA in the post administration sample or samples; and (vi) altering the administration of the agent to the subject accordingly. For example, decreased administration of the agent may be desirable to decrease expression or activity of marker to lower levels than detected, i.e., to decrease the effectiveness of the agent. According to such an embodiment, marker expression or activity may be used as an indicator of the effectiveness of an agent, even in the absence of an observable phenotypic response.

C. Methods of Treatment

The present invention provides for both prophylactic and therapeutic methods of treating a subject at risk for (or susceptible to) organ damage from reperfusion associated with aberrant marker expression or activity. With regards to both prophylactic and therapeutic methods of treatment, such treatments may be specifically tailored or modified, based on knowledge obtained from the field of pharmacogenomics. “Pharmacogenomics”, as used herein, includes the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype”, or “drug response genotype”.) Thus, another aspect of the invention provides methods for tailoring an individual's prophylactic or therapeutic treatment with either the marker molecules of the present invention or marker modulators according to that individual's drug response genotype. Pharmacogenomics allows a clinician or physician to target prophylactic or therapeutic treatments to subjects who will most benefit from the treatment and to avoid treatment of subjects who will experience toxic drug-related side effects.

1. Prophylactic Methods

In one aspect, the invention provides a method for preventing in a subject, organ damage from reperfusion associated with abnormally increased marker expression or activity, by administering to the subject a marker protein or an agent that modulates marker protein expression or at least one marker protein activity. Subjects at risk for a disease that is caused or contributed to by increased marker expression or activity can be identified by, for example, any or a combination of diagnostic or prognostic assays as described herein. Administration of a prophylactic agent can occur prior to the manifestation of symptoms characteristic of the differential marker protein expression, such that organ damage from reperfusion is prevented or, alternatively, delayed in its progression. Depending on the type of marker aberrancy (e.g., typically an increase outside the normal standard deviation), for example, a marker protein, marker protein agonist or marker protein antagonist agent can be used for treating the subject. The appropriate agent can be determined based on screening assays described herein.

2. Therapeutic Methods

Another aspect of the invention pertains to methods of modulating marker protein expression or activity for therapeutic purposes. Accordingly, in an exemplary embodiment, the modulatory method of the invention involves contacting a cell with a marker protein or agent that modulates one or more of the activities of a marker protein activity associated with the cell. An agent that modulates marker protein activity can be an agent as described herein, such as a nucleic acid or a protein, a naturally occurring target molecule of a marker protein (e.g., a marker protein substrate), a marker protein antibody, a marker protein agonist or antagonist, a peptidomimetic of a marker protein agonist or antagonist, or other small molecule. In one embodiment, the agent stimulates one or more marker protein activities. Examples of such stimulatory agents include active marker protein and a nucleic acid molecule encoding marker protein that has been introduced into the cell. In another embodiment, the agent inhibits one or more marker protein activities. Examples of such inhibitory agents include antisense marker protein nucleic acid molecules, anti-marker protein antibodies, and marker protein inhibitors. These modulatory methods can be performed in vitro (e.g., by culturing the cell with the agent) or, alternatively, in vivo (e.g., by administering the agent to a subject). As such, the present invention provides methods of treating an individual at risk for organ damage or severe reperfusion injury characterized by aberrant expression or activity of a marker protein or nucleic acid molecule. In one embodiment, the method involves administering an agent (e.g., an agent identified by a screening assay described herein), or combination of agents that modulates (e.g., upregulates or downregulates) marker protein expression or activity. In another embodiment, the method involves administering a marker protein or nucleic acid molecule as therapy to compensate for reduced or aberrant marker protein expression or activity.

Stimulation of marker protein activity is desirable in situations in which marker protein is abnormally downregulated and/or in which increased marker protein activity is likely to have a beneficial effect. For example, stimulation of marker protein activity is desirable in situations in which a marker is downregulated and/or in which increased marker protein activity is likely to have a beneficial erect. Likewise, particularly with regards to the markers listed in Tables 3-7 that are abnormally or close-to-abnormally upregulated in association with reperfusion injury, inhibition of marker protein activity is likely to have a beneficial effect.

3. Pharmacogenomics

The marker protein and nucleic acid molecules of the present invention, as well as agents, inhibitors or modulators which have a stimulatory or inhibitory effect on marker protein activity (e.g., marker gene expression) as identified by a screening assay described herein can be administered to individuals to treat (prophylactically or therapeutically) marker-associated organ damage (e.g., resulting from ischemia or reperfusion) associated with aberrant marker protein activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a marker molecule or marker modulator as well as tailoring the dosage and/or therapeutic regimen of treatment with a marker molecule or marker modulator.

Pharmacogenomics deals with clinically significant hereditary variations in the response to drugs due to altered drug disposition and abnormal action in affected persons. See, for example, Eichelbaum et al. (1996) Clin. Exp. Pharmacol. Physiol. 23(10-11):983-985 and Linden et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically substantial number of subjects taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a marker protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer and poor metabolizer. The prevalence of poor metabilizer phenotypes is different among different populations. For example, the gene coding for CYP2D6 is highly polymorphic and several mutations have been identified in poor metabilizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabilizers show no therapeutic response, as demonstrated for the analgesic effect of codeine mediated by its CYP2D6-formed metabolite morphine. The other extremes are the so called ultra-rapid metabolizers, subjects that do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a marker molecule or marker modulator of the present invention) can give an indication of whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a marker molecule or marker modulator, such as a modulator identified by one of the exemplary screening assays described herein.

This invention is further illustrated by the following examples, which should not be construed as limiting. The contents of all references, patents and published patent applications cited throughout this application, as well as the Figures and Tables, are incorporated herein by reference.

### EXAMPLES

### Example 1

### Identification and Characterization of Marker cDNA in Primate Model of Ischemia and Reperfusion Injury

A. Development of Ischemia and Reperfusion Injury in Rhesus Monkey

Three renal allografts were performed using MHC mismatched donors-recipient pairs. Donor-recipient pairs were selected based on genetic nonidentity at MHC class II. This was established based on denaturing gel electrophoresis and direct sequencing of the second exon of HLA DR B. T cell responsiveness of the recipient towards the donor was confirmed in vitro for all donor-recipient pairs using the MLR assay. Each animal was tested against all potential donors to establish the highest responder pairs for transplantation.

Additionally, three autografts were performed. In this case the animal serves as its own donor and, as such, no typing is performed. Native kidneys removed at the time of auto- or allo-transplantation were also evaluated.

Renal allotransplantation was performed using standard surgical techniques. Outbred juvenile rhesus monkeys, which were sero-negative for simian immunodeficiency virus and herpes B virus, were obtained from LABS of Virginia, Inc. (Yemassee, S.C.). Procedures were performed under general anesthesia. Transplantation was performed between genetically distinct donor-recipient pairs as determined by the MHC analysis described above. The animals were heparinized during organ harvest and implantation (100 units/kg). The allograft was implanted using standard microvascular techniques to create an end to side ansastamosis between the donor renal artery and recipient distal aorta as well as the donor renal vein and recipient vena cava. A primary ureteroneocystostomy was then created. Bilateral native nephrectomy was completed prior to closure. The procedure lasts approximately 2-3 hours.

The animals were not treated in any way for allograft rejection. It is known that untreated allografts undergo rejection while untreated autografts do not undergo rejection, but are still subject to the changes associated with surgical manipulation of the kidney. Native nontransplanted kidneys were neither rejected nor subject to surgical trauma. As such, the native kidneys give a baseline for genes that are naturally expressed in a normal kidney; autografts express these genes and have unique gene expression patterns altered not only by surgical manipulation, but also by immune rejection.

B. Obtaining Samples for Array Hybridization and Detection of Fluorescence

Needle biopsies (20 gauge) were obtained prior to kidney manipulation, at the time of graft reperfusion (approximately 30 minutes after reperfusion), and on postoperative day 3. On day 7 (or earlier if the animals rejected earlier), the animals were euthanized, and the entire kidney was processed for RNA procurement. The kidney tissue was snap frozen in liquid nitrogen, and shipped on dry ice to Wyeth-Ayerst Research in Andover, Mass. for analysis. Spleen and node tissue was also snap-frozen in liquid nitrogen and shipped in similar fashion. A wedge of kidney was submitted for immunohistochemical analysis and routine histology. The discarded normal untransplanted kidney available from each transplant procedure was also processed for histological and genetic analysis to serve as additional control tissue.

Total RNA was isolated using the RNEASY® mini kit (Quiagen, Hilden, German). Double stranded cDNA was then synthesized from total RNA using cDNA primers containing the binding site for T7 RNA polymerase. The resulting cDNA molecules were therefore tagged with the T7 promoter at the 3′ end and used as the template for in vitro transcription reaction (ITR), resulting in amplification and labeling of anti-sense RNA.

The procedure used for the cDNA synthesis followed the recommended procedure of the BRL SUPERSCRIPT® II cDNA kit. PCR was conducted (one cycle at 70° C., 10 min; cycle two at 50° C. for 62 min; and cycle three at 15.8° C. for 125 min; at 4° C. between cycles). A first strand reagent cocktail containing 1 μl RNase Inhibitor (BRL cat# 10777-019), 4 μl 5× First strand buffer, 2 μl 100 mM DTT, and 1 μl 10 mM dNTP was prepared (all from BRL cat# 8090RT). Separately, 2 μl of T7-tagged oligo-dT primer was annealed to 2 μg/9 μl RNA (11 μl reaction volume) at 70° C. for 10 minutes then held at 4° C. The annealed primer/template RNA mixture was then brought to 50° C. for 2 minutes and the first strand reagent cocktail added. This first stand reaction mix was incubated at 50° C. for 60 minutes then held at 4° C. A Second Strand synthesis cocktail containing 91 μl RNAse free water, 30 μl 5× Second strand buffer, 3 μl 10 mM dNTP's, 1 μl E. Coli Ligase, 1 μl E. Coli RNAseH and 4 μl E. Coli Polymerase was prepared on ice. The second strand reagent cocktail was the first strand reaction mixture at 4° C. and mixed by pipetting up and down. This second strand reaction mix was incubated at 15.8° C. for 2 hours. To polish off the second strand synthesis, 2 μl of T4 polymerase was then added, and incubated an additional 5 minutes. cDNA was extracted with phenol:chloroform:isoamyl alcohol (25:24:1), separated on Phase Lock Gel tube (Fisher # NC9753826), centrifuged at room temperature for 5 min at 13,000× g and precipitated with 75 μl of 7.5M NH4OAc and 375 μl absolute ethanol. After mixing and centrifugation, the resulting pellet the pellet was washed twice with 70% ethanol and resuspended in 20 μl of RNAse free water.

One half of the cDNA reaction product was used as the template for transcription in the following in vitro transcription (IVT) reaction mix. Duplicate IVT reaction mixes were prepared per sample, each containing 10 μl cDNA reaction product, 6 μl 10× Ambion reaction buffer (Ambion Cat #8151G), 6 μl NTP mix (Pharmacia Cat #27-2025-01), 3 μl 100 mM DTT, 2.4 μl 10 mM Bio-11-UTP (Enzo Cat #42815), 2.4 μl 10 mM Bio-11-CTP (Enzo Cat #42818), 2 μl RNAse Inhibitor (Ambion Cat #2684), 2 μl T7 Polymerase (Epicentre Cat # TU 950) and RNAse free water to bring final reaction volume to 60 μl. Each IVT reaction was incubated at 37° C. overnight and purified by QIAGEN® columns to remove unincorporated nucleotides as per the manufacturer's protocol. Labeled RNA was then concentrated by elution with RNAse free water and spinning at >8000× g. The yield was determined by OD₂₆₀.

C. Array Hybridization and Detection of Fluorescence

Amplified, biotin labeled RNA from the in vitro transcription reaction was ready to be hybridized to the GENECHIPs®. Since the chips were designed with 20 or 25 base oligonucleotides, the IVT RNA was fragmented in the presence of heat and Mg++ to more closely approach the size of the oligos. Once the chips were hybridized, they were washed on the automated Affymetrix fluidics machine, stained with a streptavidin-phycoerytrin conjugate and scanned.

The IVT product (biotinylated cRNA) was aliquoted at 15 mg per chip for fragmentation. The volume was adjusted to 32 ml with RNAse-free water and 8 ml of 5× Fragmentation Buffer (6.06 g Tris Base in 175 ml DEPC water, pH to 8.1 with Glacial Acetic Acid, 12.3 g Potassium Acetate, 8.04 g Magnesium Acetate, final pH˜8.4) was added. The sample was mixed, then heated to 94° C. for 35 min. The probe hybridization mix was prepared by adding 150 μl 2× MES hybridization buffer, 3 μl Acetylated BSA (50 mg/ml) (BRL #15561-020), 3 μl HS DNA (10 mg/ml), 30 μl Bio 948 oligo (500 pM), 15 μl Standard curve pool, 59 μl Rnase free water to the fragmented 40 μl IVT product, for a final probe volume of 300 μl. This probe hybridization mix was then heated to 99° C. for 5 min, then 45° C. for 5 min, touch spun at full speed in an eppifuge, then incubated overnight at 45° C. with three glass beads (Fisher #11-311A). An Affymetrix FL6800 and Affymetrix U95B GENECHIPs® composed of oligonucleotide arrays of human genes (MicroArray, Affymetrix, cat. No. 510137) were pre-hybridized with 1× MES hybridization buffer. The probe was transferred off the beads to a fresh tube. Before applying the probe to each GENECHIP® for hybridization, the probe was denatured at 99° C. for 5 min, then brought to 45° C. for 5 min followed by a full speed, 5 min spin in an eppifuge. 200 μl of denatured probe hybridization mix was loaded onto each Affymetrix GENECHIP® and incubated at 45° C. overnight on a “rotisserie” running at ˜60 RPM.

After hybridization incubation was complete for each GENECHIP®, (i.e., the Affymetrix FL6800 GENECHIP® and the Affymetrix U95B GENECHIP) the probe was carefully removed from each chip and saved. Each hybridized chip was immediately filled with 6× SSPET and ready for washing and staining in the Affymetrix chip washing station as per Affymetrix EukGE-WS2 protocol: 10 cycles of 2 mixes/cycle with nonstringent wash buffer (6× SSPE, 0.01% Tween-20) at 25° C. followed by 4 cycles of 15 mixes/cycle stringent wash buffer (100 mM MES, 0.1M NaCl, 0.01% Tween 20) at 50° C. The probe array was then stained for 10 min in SAPE (594 μl 1× Stain Buffer, 12 μl R-Phycoerythrin Streptavidin) at 25° C., then stained with antibody solution (590.4 ml 1× Stain buffer, 6 μl goat IgG, 2.6 μl anti-streptavidin goat antibody, biotinylated, Vector Laboratories # BA-0500) for 10 min at 25° C., and stained again for 10 min in SAPE at 25° C. The probe array was then finally washed in 15 cycles of 4 mixes/cycle with nonstringent buffer at 30° C. and stored at 25° C. The arrays were read by a HP GENEARRAY® Scanner, a scanning confocal microscope commercially available through Affymetrix, Santa Clara, Calif. The scanner uses an argon ion laser as the excitation source, with the emission detected by a photomultiplier tube through either a 530 nm bandpass filter (fluorescein), or a 560 nm longpass filter (phycoerythrin). Nucleic acids of either sense or antisense orientations can be used used in hybridization experiments. Arrays with probes for either orientation (reverse complements of each other) can be made using the same set of photolithographic masks by reversing the order of the photochemical steps and incorporating the complementary nucleotide.

D. Quantitative Analysis of Hybridization Patterns and Insensitivities

Following a quantitative scan of each array, or biochip, a grid is aligned to the image using the known dimensions of the array and the corner control regions as markers. The image is reduced to a simple text file containing position and intensity information using software developed at Affymetrix (GENECHIP® 3.0 software). This information is merged with another text file that contains information relating physical position on the array to probe sequence and the identity of the RNA and the specific part of the RNA for which the oligonucleotide probe is designed. Affymetrix technology utilizes differential hybridization against nucleotide probes that are designed as perfect matches (PM) to the target RNA compared to hybridization against oligos which have a single basepair mismatch (MM). The quantitative analysis of the hybridization results involves a simple form of pattern recognition based on the assumption that, in the presence of the target RNA, the PM probes will hybridize more strongly on average than their MM partners. The number of instances in which the PM hybridization signal is larger than the MM signal is computed along with the average of the logarithm of the PM/MM ratios for each probe set. These values are used to make a decision (using a predefined decision matrix) concerning the presence or absence of an RNA. To determine the quantitative RNA abundance, the average of the differences (PM minus MM) for each probe family is calculated. The advantage of the difference method is that signals from random cross-hybridization contribute equally, on average, to the PM and MM probes, while specific hybridization contributes more to the PM probes. By averaging the pairwise differences, the real signals add constructively while the contributions from cross-hybridization tend to cancel. When assessing the differences between two different RNA samples, the hybridization signals from side-by-side experiments on identically synthesized arrays are compared directly. The magnitude of the changes in the average of the difference (PM-MM) values is interpreted by comparison against a standard curve provided by samples containing known quantities (see Hill et al. (2000) Science 290:809). Data analysis programs developed at Affymetrix, such as the GENECHIP® 3.0 software, perform these operations automatically.

Distinct gene expression patterns emerged between the tissue of normal untransplanted kidneys and that of allograft and autograft kidney tissue as analyzed on each GENECHIP® (i.e., the Affymetrix FL6800 GENECHIP and the Affymetrix U95B GENECHIP®). Biopsies in the allograft and autograft kidneys were collected at four different points: prior to harvest, at or immediately following graft transplantation (30 to 60 minutes after reperfusion), three days after transplantation and seven days after transplantation (or earlier, if progression towards acute rejection was detected). Onset of acute rejection was determined by monitoring for signs of proteinuria (kidney damage can be measured by the amount of albumin or creatine in urine and/or blood). Each of the allograft transplants experienced onset of acute rejection by Day 7 after transplant.

In order to identify genes most likely to play a role in early mechanisms involving ischemia or reperfusion injury, genes were sought that revealed a pattern of abnormal expression in both autografts and allografts. Since it was expected that allografts would undergo abnormal expression patterns as a result of either ischemia or immunological response, autografts that also provided abnormal expression would indicate a response to the surgical manipulation that was likely independent of immune response, e.g., a response to ischemia and reperfusion. Furthermore, abnormal expression of genes at days 3 and 7 in autografts may indicate mediators of tissue change that occur prior to immune assault, and which may exacerbate the “on” signals after an allograft transplantation. The genes demonstrating abnormal or close-to-abnormal expression following transplantation 30 to 60 minutes after reperfusion in allografts and autografts are set forth in Table 3. Moreover, as validation, there are several genes that were previously known to be associated with ischemia and reperfusion, which are provided separately in Table 1. The genes listed in Tables 1-7 were obtained from the above-described experiments run on either the Affymetrix FL6800 GENECHIP® or the Affymetrix U95B GENECHIP®.

To identify genes that were abnormally expressed at day three after transplantation, the standard deviation of untransplanted kidney expression was calculated. Table 5 provides genes wherein the frequency of expression in allografts at day 3 is abnormally increased, as indicated by being outside the standard deviation of normal expression. These genes were suspected of initiating immune activation prior to full-blown immune assault. Similarly, the genes listed in Table 6 indicate genes that not only had abnormally increased levels of expression in allografts at day three (by a factor of at least 2), but that also had abnormally or close-to-abnormally increased expression levels in autografts. The trend towards abnormal expression of genes listed in Table 6 in autografts becomes more pronounced by day 7, as shown in Table 7.

The genes listed in Table 6 are of particular interest because they are also implicated in autografts at such an early stage of response, wherein the autografts reflect allograft expression trends. The implication that many of these genes initiate immune response is supported by dramatically increasing levels of expression in the same genes in allografts at day 7.

Animals receiving allograft transplants as opposed to autograft transplants developed distinct expression profiles as reflected in the expression levels of Tables 3 and 5-7. Since each of the three allografts underwent onset of acute rejection whereas the autografts did not, it is appreciated that differences in the expression profiles between the allografts and the autografts at each point in time could be exploited to characterize acute rejection and predict the possibility of its occurrence in further transplant subjects (and thereby provide a prognosis for long term organ survival).

### Example 2

### Identification and Characterization of Marker cDNA in Human Model of Ischemia and Reperfusion Injury

Biopsies from Human Kidney Transplants

In addition, to further identify targets for modulation of ischemia and reperfusion in humans, samples from five human kidney transplants were collected from living and cadaveric donor kidneys. Living donor kidney samples were biopsied prior to removal (pre-reperfusion) and following transplantation (30 to 60 minutes after reperfusion). While all pre-reperfusion biopsies were taken prior to organ harvest, the timing of the biopsies occurred both before and after clamping and resulted in unexpected variability in expression levels. Preferably, pre-reperfusion biopsies are taken prior to clamping. Cadaveric kidneys were biopsied after implantation and reperfusion (30 to 60 minutes after reperfusion). Isolation of RNA and quantitative analysis of hybridization patterns were conducted as shown in Example 1 above. Genes that were not previously associated with reperfusion injury and that expressed abnormal or close-to-abnormal levels of expression are set forth in Table 4. The genes listed in Table 4 were also expressed at abnormal or close-to-abnormal levels in rhesus monkeys. As validation, genes previously linked to ischemia, listed in Table 2 , also reflected abnormal expression as a result of reperfusion.

Other variations and modifications of this invention will be obvious to those skilled in the art. This invention is not limited except as set forth in the claims.

TABLE 1
Genes Previously Linked to Ischemia and Reperfusion Injury
Expression Levels Measured in Rhesus Monkey

| | | | | Autograft | Allograft | S.D. |
|---|---|---|---|---|---|---|
| Description | Accession No. | Name | Normal | Post | Post | Normal |
| V01512, class A, 20 probes, 20 in V01512 mRNA#2 1533-2061, Human | V01512 | FOS | 9.8 | 125.7 | 156.0 | 3.38 |
| cellular oncogene c-fos (complete sequence) | | | | | | |
| X56681, class A, 20 probes, 20 in X56681 mRNA 1311-1835, Human | X56681 | JUND | 69.8 | 119.7 | 123.5 | 22.39 |
| junD mRNA | | | | | | |
| X68277, class C, 20 probes, 20 in all_X68277 1459-1952, H. sapiens CL | X68277 | DUSP1 | 40.4 | 105.0 | 116.5 | 14.58 |
| 100 mRNA for protein tyrosine Phosphatese | | | | | | |
| U72649, class A, 20 probes, 20 in U72649 2206-2584, Human BTG2 | U72649 | BTG2 | 21.6 | 57.3 | 70.5 | 8.18 |
| (BTG2) mRNA, complete cds | | | | | | |
| M92843, class A, 20 probes, 20 in M92843 1144-1583, H. sapiens zinc | M92843 | ZFP36 | 19.0 | 56.7 | 68.5 | 13.02 |
| finger transcriptional regulator mRNA, complete cds | | | | | | |
| M62831, class A, 20 probes, 20 in M62831 mRNA 1210-1750, Human | M62831 | ETR101 | 10.6 | 42.3 | 43.0 | 4.78 |
| transcription factor ETR101 mRNA, complete cds | | | | | | |
| M83667, class A, 20 probes, 20 in M83667 mRNA 713-1143, Human NF- | M83667 | CEBPD | 22.7 | 42.0 | 42.0 | 7.40 |
| IL6-beta protein mRNA, complete cds | | | | | | |
| L49169, class A, 20 probes, 20 in L49169 mRNA 3270-3612, Human | L49169 | FOSB | 8.0 | 26.7 | 45.0 | 1.98 |
| G0S3 mRNA, complete cds | | | | | | |
| L19871, class A, 20 probes, 20 in L19871 1361-1793, Human activating | L19871 | ATF3 | 8.0 | 24.0 | 21.5 | 1.13 |
| transcription factor 3 (ATF3) mRNA, complete cds | | | | | | |
| M69043, class A, 20 probes, 20 in M69043 985-1459, Homo sapiens | M69043 | NFKBIA | 12.0 | 23.7 | 20.0 | 5.06 |
| MAD-3 mRNA encoding 1 kB-like activity, complete cds | | | | | | |
| Nuclear Factor Nf-116 | HG3494-HT3688 | CEBPB** | 12.8 | 22.0 | 26.0 | 7.00 |
| X53586, class A, 20 probes, 20 in X53586 mRNA 4766-5306, integrin | X53586 | ITGA6** | 11.4 | 21.3 | 13.0 | 3.93 |
| alpha 6 (or alpha E) protein gene extracted from Human mRNA for | | | | | | |
| integrin alpha 6 | | | | | | |
| U62015, class A, 20 probes, 20 in U62015 1475-1841, Homo sapiens | U62015 | CYR61 | 8.0 | 19.3 | 13.5 | 1.75 |
| Cyr61 mRNA, complete cds | | | | | | |
| M59465, class A, 20 probes, 20 in M59465 3867-4341, Human tumor | M59465 | TNFAIP3 | 9.0 | 17.0 | 10.5 | 3.38 |
| necrosis factor alpha inducible protein A20 mRNA, complete cds | | | | | | |
| X51345, class C, 20 probes, 20 in all_X51345 1604-1744, Human jun-B | X51345 | JUNB | 8.0 | 16.0 | 22.5 | 1.31 |
| mRNA for JUN-B protein | | | | | | |
| U15932, class A, 20 probes, 20 in U15932 1928-2294, Human dual- | U15932 | DUSP5 | 8.6 | 14.3 | 11.5 | 3.24 |
| specificity protein phosphatase mRNA, complete cds | | | | | | |
| uPA gene | X02419 | PLAU | 25.2 | 47.7 | 25.5 | 8.36 |
| Human early growth response protein 1 (hEGR1) | X52541 | EGR1 | 11.9 | 68.3 | 80.0 | 5.60 |
| Human DNA-binding protein CPBP (CPBP) | U44975 | COPEB | 9.4 | 37.0 | 26.0 | 3.93 |
| AI670862, 5000 nt 3 p utr built to coding-FRA-2 gene (X16706) | X16706 | FRA-2 | 8 | 22.33 | 16.00 | 2.92 |
| (associated with FOSL2) | | | | | | |
| AI290237, 5000 nt 3 p utr built to coding-FRA-2 gene (X16706) | X16706 | FRA-2 | 9.2 | 26.00 | 15.50 | 4.21 |
| (associated with FOSL2) | | | | | | |

TABLE 2
Genes Previously Linked to Ischemia and Reperfusin Injury
Expression Levels Measured in Humans

| | | Live donor 25 | Live donor 29, | Live donor 22, | Live donor, | Cadaveric | |
|---|---|---|---|---|---|---|---|
| Accession No. | Name | Pre-Reperfusion | Pre-Reperfusion | Pre-Reperfusion | Post-Reperfusion | donor 21 | S.D. Normal |
| AF001461 | COPEB | 5 | 10 | 28 | 52 | 140 | 12.10 |
| AF017307 | ELF3 | 4 | 5 | 5 | 12 | 31 | 0.58 |
| L19871 | ATF3 | 3 | 5 | 12 | 45 | 111 | 4.73 |
| Y11307 | CYR61 | 4 | 5 | 11 | 15 | 13 | 3.79 |
| X51345 | JUNB | 6 | 7 | 11 | 49 | 45 | 2.65 |
| L49169 | FOSB | 8 | 9 | 21 | 74 | 83 | 7.23 |
| U15932 | DUSP5 | 2 | 4 | 2 | 5 | 9 | 1.15 |
| M59465 | TNFAIP3 | 4 | 4 | 4 | 8 | 10 | 0.00 |
| V01512 | FOS | 12 | 10 | 19 | 166 | 237 | 4.73 |
| M62831 | ETR101 | 32 | 19 | 30 | 235 | 244 | 7.00 |
| M69043 | NFKBIA | 12 | 11 | 31 | 58 | 54 | 11.27 |
| M92843 | ZFP36 | 8 | 12 | 18 | 96 | 216 | 5.03 |
| U72649 | BTG2 | 15 | 9 | 32 | 40 | 97 | 11.93 |
| M83667 | CEBPD | 19 | 20 | 33 | 81 | 138 | 7.81 |
| X02419 | PLAU | 10 | 8 | 20 | 13 | 9 | 6.43 |
| X68277 | DUSP1 | 7 | 7 | 13 | 78 | 111 | 3.46 |
| X56681 | JUND | 64 | 60 | 119 | 118 | 143 | 32.97 |

TABLE 3
Genes Abnormally Expressed 30-60 Minutes Post Reperfusion in Autografts and Allografts of Rhesus Monkey

| | | | | Autograft | Allograft | S.D. |
|---|---|---|---|---|---|---|
| Description | Accession No. | Name | Normal | Post | Post | Normal |
| Human helix-loop helix protein (1d-2) mRNA, complete cds | M97796 | ID2 | 24.3 | 41.0 | 38.0 | 7.59 |
| TGF-beta superfamily protein | AB000584 | PLAB | 12.3 | 33.0 | 38.5 | 10.95 |
| Human TR3 orphan receptor | L13740 | NR4A1 | 8.9 | 32.0 | 41.0 | 3.50 |
| IEX-1 = radiation-inducible immediate-early gene | S81914 | IER3 | 13.6 | 29.7 | 25.5 | 7.23 |
| replication protein A | L07493 | RPA3 | 9.9 | 20.0 | 12.0 | 2.93 |
| Human epithelial-specific transcription factor ESE-1b (ESE-1) | U73843 | ELF3 | 10.6 | 18.0 | 12.0 | 4.62 |
| Human ras-related rho mRNA | M12174 | ARHB | 8.8 | 15.3 | 14.0 | 3.14 |
| Human mitogen induced nuclear orphan receptor (MINOR) | U12767 | NR4A3 | 8.0 | 14.0 | 10.5 | 1.55 |
| H16294, GENESEQN:AAF33222 hgs patent | GENESEQN: | UNK_H16294 | 11.2 | 39.33 | 31.00 | 5.81 |
| | AAF33222 | | | | | |
| AI887641, 5 p end, Kruppel-like zinc finger protein | AF001461 | ZF9 | 18 | 74.00 | 58.50 | 5.57 |
| AI439109, G-protein-coupled receptor induced protein G1G2 | AF205437 | C8FW | 9.8 | 26.00 | 19.50 | 4.76 |

TABLE 4
Genes Abnormally Expressed Before and After Ischemia
Expression Levels Measured in Humans

| | | Live donor 25 | Live donor 29, | Live donor 22, | Live donor, | Cadaveric | |
|---|---|---|---|---|---|---|---|
| Accession No. | Name | Pre-Reperfusion | Pre-Reperfusion | Pre-Reperfusion | Post-Reperfusion | donor 21 | S.D. Normal |
| S81914 | IEX-1 | 6 | 11 | 59 | 19 | 58 | 29.62 |
| L13740 | NR4A1 | 9 | 7 | 32 | 28 | 48 | 13.89 |
| AB000584 | PLAB | 15 | 8 | 22 | 49 | 131 | 7 |
| M12174 | ARHB | 2 | 4 | 7 | 11 | 12 | 2.52 |
| D13891 | ID2 | 14 | 9 | 34 | 24 | 24 | 13.23 |

TABLE 5
Genes Abnormally Expressed in Allografts on Day 3 Post-Transplant
Expression Levels Measured in Rhesus Monkey

| | | | | | | Allograft | |
|---|---|---|---|---|---|---|---|
| | Accession | | Allograft | Autograft | | Day 3/ | S.D. |
| Gene Description | No. | Name | Day 3 | Day 3 | Normal | Normal | Normal |
| M17733, class A, 20 probes, 20 in M17733 mRNA 13-505, | M17733 | Human thymosin | 231.0 | 158.3 | 131.8 | 1.75 | 30.5 |
| Human thymosin beta-4 mRNA, complete cds | | beta-4 | | | | | |
| X57351, class C, 12 probes, 12 in all_X57351 294-891, | X57351 | 1-8 D interferon- | 119.3 | 86.0 | 64.8 | 1.84 | 24.8 |
| Human 1-8D gene from interferon-inducible gene | | inducible | | | | | |
| family, Human 1-8D gene from interferon-inducible gene family | | | | | | | |
| X00274, class A, 20 probes, 16 in X00274exon#5 | X00274 | UNK_X00274 | 77.0 | 16.0 | 21.0 | 3.67 | 13.5 |
| 1-337:4 not in GB record, Human gene for HLA-DR | | | | | | | |
| alpha heavy chain a class II antigen (immune response | | | | | | | |
| gene) of the major histocompatibility complex (MHC) | | | | | | | |
| U78027, class A, 20 probes, 20 in U78027 mRNA#3 3-350, | U78027 | UNK_U78027 | 76.7 | 47.0 | 40.7 | 1.89 | 13.2 |
| L44L gene (L44-like ribosomal protein) extracted | | | | | | | |
| from Human Bruton tyrosine kinase (BTK), alpha-D- | | | | | | | |
| galactosidase A (GLA), L44-like ribosomal protein | | | | | | | |
| (L44L) and FTP3 (FTP3) genes, complete cds, L44L | | | | | | | |
| gene (L44-like ribosomal protein) extracted from | | | | | | | |
| Human Bruton tyrosine kinase (BTK), alpha-D- | | | | | | | |
| galactosidase A (GLA), L44-like ribosomal protein | | | | | | | |
| (L44L) and FTP3 (FTP3) genes, complete cds | | | | | | | |
| X06985, class A, 20 probes, 20 in X06985 mRNA 943-1393, | X06985 | HMOX1 | 72.7 | 19.0 | 21.8 | 3.33 | 15.3 |
| Human mRNA for heme oxygenase | | | | | | | |
| X02761, class C, 20 probes, 20 in all_X-02761 7082-7646, | X02761 | FN1 | 67.3 | 17.3 | 28.2 | 2.39 | 9.6 |
| Human mRNA for fibronectin (FN precursor) | | | | | | | |
| X64707, class C, 20 probes, 20 in all_X64707 401-888, | X64707 | RPL13 | 58.3 | 31.3 | 33.5 | 1.74 | 11.0 |
| H. sapiens BBC-1 mRNA | | | | | | | |
| J03040, class A, 20 probes, 20 in J03040 1508-2000, | J03040 | SPARC | 57.3 | 18.0 | 16.2 | 3.55 | 5.3 |
| Human SPARC/osteonectin mRNA, complete cds | | | | | | | |
| U93205, class A, 20 probes, 20 in U93205 588-1020, | U93205 | CLIC1 | 55.7 | 39.7 | 32.5 | 1.71 | 8.2 |
| Human nuclear chloride ion channel protein (NCC27) | | | | | | | |
| mRNA, complete cds | | | | | | | |
| M33600, class A, 20 probes, 20 in M33600 581-1109, | M33600 | HLA-DRB1 | 55.7 | 20.0 | 25.5 | 2.18 | 10.3 |
| Human MHC class II HLA-DR-beta-1 (HLA-DRB1) | | | | | | | |
| mRNA, complete cds | | | | | | | |
| Monocyte Chemotactic Protein 1 | HG4069- | SCYA2 | 55.7 | 10.0 | 10.0 | 5.57 | 4.0 |
| | HT4339 | | | | | | |
| Z74615, class C, 20 probes, 20 in all_Z74615 5320-5852, | Z74615 | COL1A1 | 52.3 | 24.3 | 22.5 | 2.33 | 8.2 |
| H. sapiens mRNA for prepro-alpha1(1) collagen | | | | | | | |
| X53331, class C, 20 probes, 20 in all_X53331 31-590, | X53331 | MGP | 51.7 | 45.3 | 26.2 | 1.97 | 12.0 |
| Human mRNA for matrix Gla protein | | | | | | | |
| X02152, class C, 20 probes, 20 in all_X02152 1090-1625, | X02152 | LDHA | 51.0 | 15.7 | 13.5 | 3.78 | 5.8 |
| Human mRNA for lactate dehydrogenase-A | | | | | | | |
| (LDH-A, EC 1.1.1.27) | | | | | | | |
| Tropomyosin Tm30 nm, Cytoskeletal | HG3514- | UNK_X04588 | 50.3 | 30.0 | 30.5 | 1.65 | 11.3 |
| | HT3708 | | | | | | |
| Z74616, class C, 20 probes, 20 in all_Z74616 4470-4992, | Z74616 | COL1A2 | 48.0 | 10.0 | 10.0 | 4.80 | 3.1 |
| H. sapiens mRNA for prepro-alpha2(I) collagen | | | | | | | |
| M34455, class A, 20 probes, 20 in M34455 1427-1889, | M34455 | INDO | 44.7 | 10.0 | 10.0 | 4.47 | 2.9 |
| Human interferon-gamma-inducible indoleamine 2,3- | | | | | | | |
| dioxygenase (IDO) mRNA, complete cds | | | | | | | |
| D13748, class A, 20 probes, 20 in D13748 812-1352, | D13748 | EIF4A1 | 41.7 | 20.0 | 21.3 | 1.95 | 8.2 |
| Human mRNA for eukaryotic initiation factor 4AI | | | | | | | |
| J04456, class A, 20 probes, 20 in J04456 31-469, | J04456 | LGALS1 | 41.0 | 10.7 | 10.0 | 4.10 | 2.9 |
| Human 14 kd lectin mRNA, complete cds | | | | | | | |
| L20688, class A, 20 probes, 20 in L20688 864-1188, | L20688 | ARHGDIB | 38.7 | 19.7 | 20.7 | 1.87 | 10.0 |
| Human GDP-dissociaion inhibitor protein (ly-GDI) | | | | | | | |
| mRNA, complete cds | | | | | | | |
| X52022, class A, 20 probes, 20 in X52022 9941-10349, | X52022 | COL6A3 | 38.3 | 10.0 | 10.0 | 3.83 | 1.7 |
| H. sapiens RNA for type VI collagen alpha3 chain | | | | | | | |
| Decorin, Alt. Splice 1 | HG3431- | DCN | 38.0 | 13.0 | 10.5 | 3.62 | 5.5 |
| | HT3616 | | | | | | |
| U70439, class A, 20 probes, 20 in U70439 956-1407, | U70439 | UNK_U70439 | 37.7 | 22.3 | 23.3 | 1.61 | 3.9 |
| Human silver-stainable protein SSP29 mRNA, complete cds | | | | | | | |
| M83751, class A, 20 probes, 20 in M83751 539-1013, | M83751 | ARP | 37.0 | 24.0 | 20.3 | 1.82 | 8.9 |
| Human arginine-rich protein (ARP) gene, complete cds | | | | | | | |
| U32944, class A, 20 probes, 20 in U32944 162-540, | U32944 | PIN | 34.7 | 19.7 | 16.7 | 2.08 | 7.8 |
| Human cytoplasmic dynein light chain 1 (hd1c1) | | | | | | | |
| mRNA, complete cds | | | | | | | |
| X82456, class C, 20 probes, 20 in all_X82456 3287-3834, | X82456 | LASP1 | 33.7 | 19.7 | 18.3 | 1.84 | 7.2 |
| H. sapiens MLN50 mRNA | | | | | | | |
| D10522, class A, 20 probes, 20 in D10522 2000-2546, | D10522 | MACS | 33.3 | 10.0 | 10.0 | 3.33 | 3.1 |
| Human mRNA for 80 K-L protein, complete cds | | | | | | | |
| SS4005, class A, 20 probes, 20 in S54005 2-197, | S54005 | TMSB10 | 33.0 | 15.0 | 10.0 | 3.30 | 5.5 |
| thymosin beta-10 [human, metastatic melanoma cell | | | | | | | |
| line, mRNA, 453 nt] | | | | | | | |
| L00389, class C, 20 probes, 20 in all_L00389 1196-1792, | L00389 | CYP1A2 | 32.7 | 18.3 | 18.0 | 1.81 | 41.7 |
| Human cytochrome P-450 4 gene | | | | | | | |
| U50523, class A, 20 probes, 20 in U50523 858-1344, | U50523 | ARPC2 | 32.3 | 15.0 | 14.2 | 2.28 | 4.6 |
| Human BRCA2 region, mRNA sequence CG037 | | | | | | | |
| D45248, class A, 20 probes, 20 in D45248 389-773, | D45248 | PSME2 | 32.3 | 13.7 | 13.8 | 2.34 | 6.6 |
| Human mRNA for proteasome activator hPA28 subunit | | | | | | | |
| beta, complete cds | | | | | | | |
| X02530, class C, 20 probes, 20 in all_X02530 571-1118, | X02530 | SCYB10 | 32.3 | 10.0 | 10.0 | 3.23 | 4.4 |
| Human mRNA for gamma-interferon inducible | | | | | | | |
| early response gene (with homology to platelet proteins) | | | | | | | |
| M26576, class B, 20 probes, 10 in M26576exon 43-289: | M26576 | COL4A1 | 31.7 | 20.3 | 16.2 | 1.96 | 4.1 |
| 10 not in GB record, COL4A1 gene (alpha-1 type IV | | | | | | | |
| collagen) extracted from Human alpha-1 collagen type | | | | | | | |
| IV gene | | | | | | | |
| Nuclear Factor Nf-116 | HG3494- | CEBPB | 30.7 | 10.0 | 11.2 | 2.75 | 6.4 |
| X15187, class B, 20 probes, 10 in X15187cds 2089-2380: | X15187 | TRA1 | 30.3 | 12.7 | 14.7 | 2.07 | 5.0 |
| 10 in reverseSequence, 2521-2737, Human tra 1 | | | | | | | |
| mRNA for human homologue of murine tumor rejection | | | | | | | |
| antigen gp96 | | | | | | | |
| Fibronectin, Alt. Splice 1 | HG3044- | FN1 | 29.7 | 10.0 | 11.7 | 2.54 | 3.9 |
| | HT3742 | | | | | | |
| U22431, class A, 20 probes, 20 in U22431 3070-3644, | U22431 | HIF1A | 29.0 | 10.0 | 10.0 | 2.90 | 3.2 |
| Human hypoxia-inducible factor 1 alpha (HIF-1 alpha) | | | | | | | |
| mRNA, complete cds | | | | | | | |
| X06700, class C, 20 probes, 20 in all_X06700 1946-2466, | X06700 | COL3A1 | 29.0 | 10.0 | 10.0 | 2.90 | 3.0 |
| Human mRNA 3′ region for pro-alpha1(III) | | | | | | | |
| collagen | | | | | | | |
| M31166, class A, 20 probes, 20 in M31166mRNA | M31166 | PTX3 | 28.7 | 10.0 | 10.0 | 2.87 | 1.2 |
| 1286-1784, Human tumor necrosis factor-inducible | | | | | | | |
| (TSG-14) mRNA, complete cds | | | | | | | |
| AB001325, class A, 20 probes, 20 in AB001325 967-1387, | AB001325 | AQP3 | 28.0 | 10.3 | 13.2 | 2.13 | 3.4 |
| Human AQP3 gene for aquaporine 3 (water | | | | | | | |
| channel), partail cds | | | | | | | |
| M63573, class A, 20 probes, 20 in M63573 370-802, | M63573 | PPIB | 27.7 | 11.3 | 10.0 | 2.77 | 6.1 |
| Human secreted cyclophilin-like protein (SCYLP) | | | | | | | |
| mRNA, complete cds | | | | | | | |
| X01703, class A, 20 probes, 20 in X01703exon#4 929-1151, | X01703 | TUBA3 | 27.3 | 10.0 | 10.0 | 2.73 | 3.9 |
| Human gene for alpha-tubulin (b alpha 1) | | | | | | | |
| U03057, class A, 20 probes, 20 in U03057 2172-2724, | U03057 | SNL | 25.3 | 10.0 | 10.0 | 2.53 | 6.3 |
| Human actin bundling protein (HSN) mRNA, complete cds | | | | | | | |
| D38583, class A, 20 probes, 20 in D38583 109-475, | D38583 | S100A11 | 25.3 | 11.0 | 10.0 | 2.53 | 4.7 |
| Human mRNA for calgizzarin, complete cds | | | | | | | |
| U52101, class A, 20 probes, 20 in U52101 61-451, | U52101 | EMP3 | 24.3 | 10.0 | 10.0 | 2.43 | 1.8 |
| Human YMP mRNA, complete cds | | | | | | | |
| X13334, class A, 20 probes, 19 in X13334cds 659-1049: | X13334 | CD14 | 22.0 | 10.0 | 10.0 | 2.20 | 1.2 |
| 1 in reverseSequence, 1234, Human CD14 | | | | | | | |
| mRNA for myelid cell-specific leucine-rich | | | | | | | |
| glycoprotein | | | | | | | |

TABLE 6
Genes Differentially Expressed in Allografts on Day 3 Post - Transplant
Expression Levels Measured in Rhesus Monkey
(Autografts Trend Toward Allograft Levels)

| | Accession | | Allograft | Autograft | | Allograft | S.D. |
|---|---|---|---|---|---|---|---|
| Gene Description | No. | Name | Day 3 | Day 3 | Normal | Day 3/Normal | Normal |
| M95787, class A, 20 probes, 20 in M95787 494-1004, | M95787 | TAGLN | 87.0 | 43.0 | 30.3 | 2.87 | 14.8 |
| Human 22kDa smooth muscle protein (SM22) mRNA, | | | | | | | |
| complete cds | | | | | | | |
| D00017, class A, 20 probes, 20 in D00017 851-1319, | D00017 | ANXA2 | 75.7 | 43.0 | 35.3 | 2.14 | 10.5 |
| Human lipocortin II mRNA | | | | | | | |
| J03801, class A, 20 probes, 20 in J03801 911-1418, | J03801 | LYZ | 70.3 | 28.7 | 35.2 | 2.00 | 8.4 |
| Human lysozyme mRNA, complete cds with an Alu | | | | | | | |
| repeat in the 3′ flank | | | | | | | |
| M19045, class A, 20 probes, 20 in M19045 907-1414, | M19045 | LYZ | 67.0 | 38.3 | 31.3 | 2.14 | 7.6 |
| Human lysozyme mRNA, complete cds | | | | | | | |
| X14008, class A, 20 probes, 20 in X14008mRNA 926-1433, | X14008 | LYZ | 64.0 | 34.0 | 27.8 | 2.30 | 6.3 |
| Human lysozyme gene (EC 3.2.1.17) | | | | | | | |
| L15702, class A, 20 probes, 20 in L15702 1778-2279, | L15702 | BF | 57.0 | 27.7 | 12.7 | 4.50 | 4.2 |
| Human complement factor B mRNA, complete cds | | | | | | | |
| X65965, class A, 18 probes, 18 in X65965exon#1-2 | X65965 | UNK_X65965 | 56.0 | 18.0 | 11.7 | 4.80 | 3.9 |
| 32-94, H. sapiens SOD-2 gene for manganese | | | | | | | |
| superoxide dismutase./gb = X65965/ntype = DNA | | | | | | | |
| /annot = exon | | | | | | | |
| X13839, class C, 20 probes, 20 in all_X13839 768-1300, | X13839 | ACTA2 | 49.3 | 29.7 | 18.0 | 2.74 | 15.7 |
| Human mRNA for vascular smooth muscle | | | | | | | |
| alpha-actin | | | | | | | |
| M59815, class A, 20 probes, 20 in M59815mRNA | M59815 | C4A | 31.0 | 30.7 | 10.3 | 3.00 | 5.6 |
| 5022-5424, Human complement component C4A gene | | | | | | | |
| M32053, class C, 20 probes, 20 in all_M32053 2900-3489, | M32053 | UNK_M32053 | 27.3 | 21.0 | 10.3 | 2.65 | 2.9 |
| Human H19 RNA gene, complete cds (spliced in | | | | | | | |
| silico) | | | | | | | |
| M38591, class A, 20 probes, 20 in M38591 120-600, | M38591 | S100A10 | 26.7 | 19.7 | 12.7 | 2.11 | 3.8 |
| Homo sapiens cellular ligand of annexin II (p11) | | | | | | | |
| mRNA, complete cds | | | | | | | |

TABLE 7
Genes Abnormally Expressed in Allografts on Day 7 Post-Transplant
Expression Levels Measured in Rhesus Monkey
(Autografts Trend Toward Allograft Levels)

| | | | | | | S.D. | Autograft |
|---|---|---|---|---|---|---|---|
| | Accession | | Allograft | Autograft | Normal | Normal | Day 7 |
| Name | No. | Description | Day 7 | Day 7 | Day 7 | Day 7 | Abnormal? |
| TMSB4X | M17733 | Human thymosin beta-r mRNA, complete cds | 360.78 | 203.71 | 131.14 | 26.30 | YES |
| FTH1 | L20941 | Human ferritin Heavy chain mRNA, complete cds | 203.56 | 113.00 | 96.71 | 38.64 | |
| MYL6 | HG2815-HT2931 | Myosin, Light Chain, Alkali, Smooth Muscle, Non- | 222.44 | 119.00 | 94.00 | 20.03 | YES |
| | | Muscle, Alt. Splice 2 | | | | | |
| MT1H | X64177 | H. sapiens mRNA for metallothionein | 165.22 | 226.00 | 79.29 | 39.20 | YES |
| HLA-A | M94880 | Human MHC class I (HLA-A*8001) mRNA | 342.11 | 96.71 | 74.14 | 34.07 | |
| HNRPA1 | X12671 | hnrnp a1 protein gene extracted from Human gene for | 143.67 | 92.71 | 70.43 | 13.19 | YES |
| | | heterogeneous nuclear ribonucleoprotein (hnRNP) | | | | | |
| | | core protein A1 | | | | | |
| PSAP | J03077 | Human co-beta glucosidase (proactivator) mRNA, | 155.67 | 84.00 | 67.86 | 19.18 | |
| | | complete cds | | | | | |
| IFITM2 | X57351 | Human 1-8D gene from interferon-inducible gene | 160.33 | 79.57 | 64.86 | 18.60 | |
| | | family, Human 1-8D gene from interferon-inducible | | | | | |
| | | gene family | | | | | |
| ACTG1 | M19283 | Human cytoskeletal gamma-actin gene, complete cds | 190.67 | 88.57 | 62.57 | 23.01 | YES |
| RPS12 | HG613-HT613 | Ribosomal Protein S12 | 172.22 | 75.57 | 62.29 | 22.27 | |
| UNK_X12432 | HG3597-HT3800 | Major Histocompatibility Complex, Class I | 213.78 | 74.00 | 61.29 | 23.47 | |
| CST3 | M27891 | Human cystatin C (CST3) gene | 199.89 | 102.57 | 60.86 | 40.33 | |
| HMG1 | D63874 | Human mRNA for HMG-1, complete cds | 141.78 | 72.29 | 60.14 | 18.23 | |
| NPM1 | M23613 | Human nucleophosmin mRNA, complete cds | 125.33 | 72.00 | 60.00 | 12.37 | |
| UNK_V00599 | V00599 | Human mRNA fragment encoding beta-tubulin (from | 204.89 | 83.57 | 57.57 | 10.23 | YES |
| | | clone D-beta-1) | | | | | |
| HLA-A | D32129 | Human mRNA for HLA class-I (HLA-A26) heavy | 249.89 | 75.71 | 56.00 | 25.87 | |
| | | chain, complete cds (clone cMIY-1) | | | | | |
| CRYAB | S45630 | alpha B-crystallin = Rosenthal fiber component [human, | 129.67 | 83.86 | 55.14 | 20.57 | YES |
| | | glioma cell line, mRNA, 691 nt] | | | | | |
| HLA-C | HG658-HT658 | Major Histocompatibility Complex, Class I, C | 228.44 | 72.43 | 54.57 | 34.77 | |
| MT2A | V00594 | Human mRNA for metallothionein from cadmium- | 313.00 | 314.43 | 54.29 | 48.72 | YES |
| | | treated cells | | | | | |
| CACYBP | HG2788-HT2896 | Calcylin | 142.67 | 78.86 | 53.43 | 10.70 | YES |
| CD63 | X62654 | ME491 gene extracted from H. sapiens gene for | 105.78 | 63.71 | 47.86 | 16.61 | |
| | | Me491/CD63 antigen | | | | | |
| UNK_L25080 | L25080 | Human GRP-binding protein (rhoA) mRNA, complete | 94.67 | 62.57 | 46.71 | 13.90 | YES |
| | | cds | | | | | |
| HNRPA1 | X04347 | Human liver mRNA fragment DNA binding protein | 134.33 | 57.14 | 44.00 | 13.94 | |
| | | UPI homologue (C-terminus) | | | | | |
| UNK_M55998 | M55998 | Human alpha-1 collagen type I gene, 3′ end | 195.89 | 119.71 | 43.57 | 27.65 | YES |
| VIM | Z19554 | H. sapiens vimentin gene | 299.11 | 61.57 | 43.00 | 20.42 | |
| UNK_U78027 | U78027 | L44L gene (L44-like ribosomal protein) extracted | 97.67 | 57.14 | 41.00 | 9.03 | YES |
| | | from Human Bruton tyrosine kinase (BTK), alpha-D- | | | | | |
| | | galactosidase A | | | | | |
| CD81 | M33680 | Human 26-kDa cell surface protein TAPA-1 mRNA, | 87.89 | 52.71 | 39.86 | 11.14 | YES |
| | | complete cds | | | | | |
| KIAA0069 | D31885 | Human mRNA for KIAA0069 gene, partial cds | 84.11 | 59.57 | 38.43 | 13.27 | YES |
| ANXA2 | D00017 | Human lipocortin II mRNA | 135.67 | 55.71 | 35.57 | 8.18 | YES |
| B2M | S82297 | beta 2-microglobulin {11 bp deleted between | 187.67 | 51.29 | 33.14 | 5.05 | YES |
| | | nucleotides 98-99} [human, colon cancer cell line | | | | | |
| | | HCT, mRNA Mutant, 416 nt] | | | | | |
| LYZ | J03801 | Human lysozyme mRNA, complete cds with an Alu | 98.22 | 47.71 | 32.71 | 10.69 | YES |
| | | repeat in the 3′ flank | | | | | |
| HBB | M25079 | Human sickle cell beta-globin mRNA, complete cds | 96.89 | 74.00 | 30.43 | 18.69 | YES |
| HBB | HG1428-HT1428 | Globin, Beta | 95.56 | 62.29 | 30.14 | 20.75 | YES |
| LYZ | M19045 | Human lysozyme mRNA, complete cds | 84.44 | 51.29 | 29.57 | 8.89 | YES |
| TAGLN | M95787 | Human 22kDa smooth muscle protein (SM22) mRNA, | 138.78 | 106.14 | 28.71 | 13.26 | YES |
| | | complete cds | | | | | |
| MT1L | X76717 | H. sapiens MT-11 mRNA | 57.78 | 73.14 | 28.71 | 6.91 | YES |
| LYZ | X14008 | Human lysozyme gene (EC 3.2.1.17) | 84.11 | 44.86 | 26.43 | 4.97 | YES |
| MGP | X53331 | Human mRNA for matrix Gla protein | 53.56 | 69.29 | 25.14 | 9.95 | YES |
| IGF2 | HG3543-HT3739 | Insulin-Like Growth Factor 2 | 59.67 | 49.57 | 24.29 | 6.79 | YES |
| COL1A1 | Z74615 | H. sapiens mRNA for prepro-alpha(1) collagen | 62.00 | 40.71 | 22.57 | 6.74 | YES |
| CEBPD | M83667 | Human NF-IL6-beta protein mRNA, complete cds | 61.56 | 36.29 | 21.86 | 7.60 | YES |
| LGALS3 | M57710 | Human lgE-binding protein (epsilon-BP) mRNA, | 54.67 | 47.43 | 18.29 | 8.56 | YES |
| | | complete cds | | | | | |
| LASP1 | X82456 | H. sapiens MLN50 mRNA | 80.11 | 30.57 | 18.14 | 5.72 | YES |
| CSRP1 | M76378 | Human cysteine-rich protein (CRP) gene | 38.00 | 32.86 | 17.86 | 7.11 | YES |
| ACTA2 | X13839 | Human mRNA for vascular smooth muscle alpha-actin | 69.89 | 56.86 | 16.57 | 15.29 | YES |
| COL4A1 | M26576 | COL4A1 gene (alpha-1 type IV collagen) exracted | 66.56 | 36.14 | 15.86 | 4.07 | YES |
| | | from Human alpha-1 collagen type IV gene | | | | | |
| UROD | X89267 | H. sapiens DNA for uroporphyrinogen decarboxylase | 31.11 | 31.29 | 15.57 | 5.02 | YES |
| | | gene | | | | | |
| ELA1 | M16652 | Human pancreatic elastase IIA mRNA, complete cds | 35.89 | 27.14 | 15.14 | 2.23 | YES |
| BF | L15702 | Human complement factor B mRNA, complete cds | 69.67 | 42.57 | 12.71 | 3.51 | YES |
| DCN | HG3431-HT3616 | Decorin, Alt. Splice 1 | 42.89 | 25.57 | 10.14 | 4.46 | YES |
| C4A | M59815 | Human complement component C4A gene | 58.67 | 37.43 | 10.00 | 5.47 | YES |
| COL1A2 | Z74616 | H. sapiens mRNA for prepro-alpha2(I) collagen | 50.00 | 19.14 | 10.00 | 2.97 | YES |
| TUBA3 | X01703 | Human gene for alpha-tubulin (b alpha 1) | 38.67 | 23.29 | 10.00 | 3.69 | YES |
| COL3A1 | X06700 | Human mRNA 3′ region for pro-alpha 1(III) collagen | 29.00 | 20.14 | 10.00 | 2.66 | YES |
| MT1G | J03910 | Human (clone 14VS) metallothionein-IG (MT1G) | 20.56 | 42.00 | 10.00 | 3.43 | YES |
| | | gene, complete cds | | | | | |
| CASP4 | U28014 | Human cysteine protease (ICErel-II) mRNA, | 20.44 | 15.29 | 10.00 | 1.17 | YES |
| | | complete cds | | | | | |

1-26. (canceled)

27. A method of screening for a compound capable of treating or preventing organ damage resulting from reperfusion comprising the steps of:
(a) contacting a sample comprising a marker selected from the group consisting of the markers listed in Table 3 with a test compound; and
(b) determining whether the level of expression or activity of the marker is decreased relative to the level of expression or activity of the marker in a sample not contacted with the compound,
wherein a decrease in the level of expression or activity of the marker in the contacted sample identifies the compound as one that is capable of treating or preventing the organ damage resulting from reperfusion.

28. A method of screening for a compound capable of treating or preventing organ damage resulting from reperfusion comprising the steps of:
(a) contacting a sample containing the nucleic acid sequence set forth in SEQ ID NO:1 and/or the amino acid sequence set forth in SEQ ID NO:2 with a test compound; and
(b) determining whether the level of expression or activity of the nucleic acid sequence set forth in SEQ ID NO:1 and/or the amino acid sequence set forth in SEQ ID NO:2 is decreased relative to the level of expression or activity of the nucleic acid sequence set forth in SEQ ID NoO:1 and/or the amino acid sequence set forth in SEQ ID NO:2 in a sample not contacted with the compound,
wherein a decrease in the level of expression or activity of the nucleic acid sequence set forth in SEQ ID NO:1 and/or the amino acid sequence set forth in SEQ ID NO:2 in the contacted sample identifies the compound as one that is capable of treating or preventing the organ damage resulting from reperfusion.

29. A method of screening for a compound capable of treating or preventing organ damage resulting from reperfusion comprising the steps of:
(a) contacting a sample comprising a marker selected from the group consisting of the markers listed in Table 3 with a test compound; and
(b) determining whether the level of expression or activity of the marker is decreased relative to the level of expression or activity of the marker in a reference sample,
wherein a decrease in the level of expression or activity of the marker in the contacted sample identifies the compound as one that is capable of treating or preventing the organ damage resulting from reperfusion.

30. A method of screening for a compound capable of treating or preventing organ damage resulting from reperfusion comprising the steps of:
(a) contacting a sample containing the nucleic acid sequence set forth in SEQ ID NO:1 and/or the amino acid sequence set forth in SEQ ID NO:2 with a test compound; and
(b) determining whether the level of expression or activity of the nucleic acid sequence set forth in SEQ ID NO:1 and/or the amino acid sequence set forth in SEQ ID NO:2 is decreased relative to the level of expression or activity of the nucleic acid sequence set forth in SEQ ID NO:1 and/or the amino acid sequence set forth in SEQ ID NO:2 in a reference sample,
wherein a decrease in the level of expression or activity of the nucleic acid sequence set forth in SEQ ID NO:1 and/or the amino acid sequence set forth in SEQ ID NO:2 in the contacted sample identifies the compound as one that is capable of treating or preventing the organ damage resulting from reperfusion.
